(19) United States
(12) Patent Application Publication
(10) Pub. No.: US 2011/0196181 A1
(43) Pub. Date: Aug. 11, 2011

(54) PROCESS FOR REACTING AN AROMATIC HYDROCARBON IN THE PRESENCE OF HYDROGEN

(75) Inventors: Michael Becker, Singapore (SG); Axel Salden, Stuttgart (DE); Bianca Stäck, Mannheim (DE); Jochem Henkelmann, Mannheim (DE); Steffen Springmann, Stuttgart (DE); Frederik van Laar, Dubai (AE); Wilhelm Ruppel, Mannheim (DE); Peter Resch, Hettenleidelheim (DE); Michael Bender, Jersey City, NJ (US)

(73) Assignee: BASF SE

(21) Appl. No.: 13/088,627

(22) Filed: Apr. 18, 2011

Related U.S. Application Data
(63) Continuation of application No. 12/158,730, filed on Jun. 23, 2008.

(30) Foreign Application Priority Data
Dec. 23, 2005 (DE) 102005062354.9

(51) Int. Cl. C07C 5/10; C07C 1/32

(52) U.S. Cl. 585/269; 585/469

(57) ABSTRACT

Processes comprising: providing a starting material comprising one or more aromatic hydrocarbons, and having an aromatic sulfur compound content and a total sulfur content; reducing the aromatic sulfur compound content and the total sulfur content in the starting material; and hydrogenating the one or more aromatic hydrocarbons in the presence of a supported ruthenium catalyst and hydrogen.

### CROSS-REFERENCE TO RELATED APPLICATIONS

This application is continuation of U.S. Ser. No. 12/158,730, filed Jun. 23, 2008, currently pending, which was a national stage application, under 35 U.S.C. §371 of PCT/EP2006/070186, filed Dec. 22, 2006, which claimed priority of German Application No. 10 2005 062 354.9, filed Dec. 23, 2005.

### BACKGROUND OF THE INVENTION

There exist numerous processes for hydrogenating benzene to cyclohexane. These hydrogenations are carried out predominantly over nickel and platinum catalysts in the liquid or gas phase (here, cf., inter alia, U.S. Pat. No. 3,597,489, U.S. Pat. No. 2,898,387, GB 799,396). Typically, the majority of the benzene is first hydrogenated to cyclohexane in a first reactor and then the unconverted amount of benzene is converted to cyclohexane in one or more downstream reactors.

The strongly exothermic hydrogenation reaction requires careful temperature and residence time control in order to achieve full conversion at high selectivity. In particular, significant formation of methylcyclopentane, which proceeds preferentially at relatively high temperatures, has to be suppressed. Typical cyclohexane specifications require a residual benzene content of <100 ppm and a methylcyclopentane content of <200 ppm. The content of n-paraffins (for example n-pentane, n-hexane) is likewise also critical. These undesired compounds are likewise formed preferentially at relatively high hydrogenation temperatures and, just like methylcyclopentane, can be removed from the desired cyclohexane only by complicated separating operations (for example extraction, rectification or use of molecular sieves, as described in GB 1,341,057). The catalyst used too has a strong influence on the degree of formation of undesired secondary components, such as methylcyclohexane, n-hexane, n-pentane, etc.

In view of this background, it is desirable to carry out the hydrogenation at minimum temperatures. On the other hand, this is limited, since, depending on the type of hydrogenation catalyst used, an adequately high hydrogenation activity of the catalyst, which is in turn sufficient for an economically viable space-time yield, is achieved only from relatively high temperatures.

The nickel and platinum catalysts used for the benzene hydrogenation additionally have a series of disadvantages. Nickel catalysts are very sensitive toward sulfur-containing impurities in benzene, so that either very pure benzene has to be used for the hydrogenation, or, as described in GB 1,104,275, a platinum catalyst which tolerates a higher sulfur content is used in the main reactor, thus protecting the postreactor which comprises a nickel catalyst.

Another possibility is to dope the hydrogenation catalyst with rhenium, as described in GB 1,155,539, or to incorporate ion exchangers into the hydrogenation catalyst, as disclosed in GB 1,144,499. However, the preparation of such catalysts is complicated and expensive.

Platinum catalysts have fewer disadvantages than nickel catalysts, but are very expensive.

As an alternative, the recent literature has therefore referred to ruthenium-containing catalysts for hydrogenating benzene to cyclohexane.

SU 319 582 describes ruthenium suspension catalysts which have been doped with palladium, platinum or rhodium for preparing cyclohexane from benzene. However, these are very expensive owing to the palladium, platinum or rhodium used, and the workup and recovery of the catalyst is additionally both complicated and expensive in the case of suspension catalysts.

U.S. Pat. No. 3,917,540 describes Al₂O₃-supported catalysts for preparing cyclohexane from benzene. As the active metal, these comprise a noble metal from transition group VIII of the Periodic Table, and also an alkali metal and technetium or rhenium. Also described in U.S. Pat. No. 3,244,644 are η-Al₂O₃-supported ruthenium hydrogenation catalysts which are also said to be suitable for hydrogenating benzene. However, these catalysts comprise at least 5% active metal. Moreover, the preparation of η-Al₂O₃ is both complicated and expensive.

In addition, WO 00/63142 describes, inter alia, the hydrogenation of unsubstituted aromatics using a catalyst which comprises, as the active metal, at least one metal of transition group VIII of the Periodic Table and which has been applied to a support having macropores. Suitable active metals are in particular ruthenium and suitable supports are in particular appropriate aluminum oxides and zirconium dioxides.

One advantage of these processes lies in the comparatively favorable costs of ruthenium which is used as the active metal for the catalyst in comparison to the costs which arise as a result of other hydrogenation metals such as palladium, platinum or rhodium. However, a disadvantage here too is that these ruthenium catalysts are sensitive toward sulfur impurities.

EP 600 406 discloses that unsaturated hydrocarbons such as alkenes (for example ethene) which are contaminated with thiophene can be desulfurized by treating the unsaturated hydrocarbon in the presence of a copper-zinc desulfurizing agent which has a copper/zinc atomic ratio of 1:about 0.3-10, and which is obtainable by a coprecipitation process, with from 0.01 to 4% by volume of hydrogen. In particular, it is emphasized that the amount of hydrogen should not exceed these values, since this leads to undesired hydrogenation of the unsaturated hydrocarbons to be purified.

The present invention relates to a process for converting an aromatic hydrocarbon which comprises aromatic sulfur compounds, or a mixture of aromatic hydrocarbons which comprises aromatic sulfur compounds, if appropriate in the presence of hydrogen, wherein, in a first step, aromatic sulfur compounds are removed (step a), and, in a second step, the aromatic hydrocarbon or the mixture of aromatic hydrocarbons is hydrogenated in the presence of a supported ruthenium catalyst in the presence of hydrogen (step b).

### BRIEF SUMMARY OF THE INVENTION

In one embodiment, the present invention relates to a process in which the aromatic hydrocarbon is benzene. In a further embodiment, the present invention relates to a process wherein a mixture of aromatic hydrocarbons is used. In this case, it is possible, for example, to use mixtures which comprise benzene and toluene. However, it is also possible to use mixtures which comprise benzene and xylene or a xylene isomer mixture, or mixtures which comprise benzene, toluene and xylene or a xylene isomer mixture. In step a), the content of aromatic sulfur compounds, for example thiophene, is lowered to ≦70 ppb, and the total sulfur content to a total of ≦200 ppb, and, in step b), the desulfurized aromatic hydrocarbon or the desulfurized mixture of aromatic hydrocarbons is reduced in the presence of a supported ruthenium catalyst and hydrogen to the corresponding cycloaliphatic hydrocarbon or the corresponding mixture of corresponding cycloaliphatic hydrocarbons. In the case of benzene, the hydrogenation product obtained is thus cyclohexane, that obtained from toluene is methylcyclohexane and that obtained from xylene is the dimethylcyclohexane corresponding in each case, and that obtained from a xylene isomer mixture is the corresponding dimethylcyclohexane isomer mixture which can be purified by distillation.

It was a primary object of the present invention to provide a process for hydrogenating aromatic hydrocarbons or mixtures thereof which comprise aromatic sulfur compounds to the corresponding cycloaliphatics or mixtures thereof, in particular benzene to obtain cyclohexane, and which enables cycloaliphatics, or the mixtures thereof, to be obtained with very high selectivity and space-time yield.

Accordingly, the present invention relates to a process for converting an aromatic hydrocarbon which comprises aromatic sulfur compounds, or a mixture of aromatic hydrocarbons which comprises aromatic sulfur compounds, wherein, in a first step, aromatic sulfur compounds, if appropriate in the presence of hydrogen, are removed (step a); this desulfurization is carried out in the presence of a copper-zinc desulfurizing agent which has a copper:zinc atomic ratio of from 1:0.3 to 1:10 and is obtainable by a coprecipitation process. In a second step, the aromatic hydrocarbon thus obtained or the mixture of aromatic hydrocarbons thus obtained is hydrogenated in the presence of a supported ruthenium catalyst and hydrogen to give the corresponding cycloaliphatics or mixtures thereof (step b), the catalyst having been applied to a support which has meso- and/or macropores.

In a preferred embodiment, the aromatic hydrocarbon used is benzene which is hydrogenated to cyclohexane in the presence of hydrogen.

In a further preferred embodiment, a mixture of aromatic hydrocarbons is used, which is hydrogenated to the corresponding mixture of cycloaliphatics in the presence of hydrogen. Useful mixtures of aromatic hydrocarbons are those which comprise benzene and toluene, or benzene and xylene or a xylene isomer mixture, or benzene, toluene and xylene or a xylene isomer mixture. The hydrogenation affords cyclohexane from benzene, methylcyclohexane from toluene, and the corresponding dimethylcyclohexanes from the xylenes.

### DETAILED DESCRIPTION OF THE INVENTION

In step a), the aromatic hydrocarbon or the mixture of aromatic hydrocarbons, each of which comprises aromatic sulfur compounds as an impurity, is desulfurized.

Possible aromatic sulfur-containing impurities are particularly thiophene, benzothiophene, dibenzothiophene or corresponding alkylated derivatives, in particular thiophene. In addition to these aromatic sulfur compounds, it is also possible for further sulfur-containing impurities, for example hydrogen sulfide, mercaptans such as methyl mercaptan, tetrahydrothiophene, disulfides such as dimethyl disulfide, COS or CS₂, referred to hereinafter as nonaromatic sulfur compounds, to be present in the aromatic hydrocarbon or the mixture of aromatic hydrocarbons. In addition, other impurities may also be present, such as water, C₅-C₇-alkanes, for example n-heptane, C₅-C₇-alkenes, for example pentene or hexene, where the double bond may be present at any position in the carbon skeleton, C₅-C₇-cycloalkanes, for example methylcyclopentane, ethylcyclopentane, dimethylcyclopentane, cyclohexane, methylcyclohexane, or C₅-C₇-cycloalkenes, for example cyclohexene.

The aromatic hydrocarbon used in a particular embodiment generally has a purity of >98% by weight, in particular >99% by weight, preferably >99.5% by weight, especially preferably >99.9% by weight. When a mixture of aromatic hydrocarbons is used, the fraction of aromatic hydrocarbons in the mixture used is >98% by weight, in particular >99% by weight, preferably >99.5% by weight, especially preferably >99.9% by weight. In both cases, the content of aromatic sulfur-containing impurities may be up to 2 ppm by weight, preferably up to 1 ppm by weight. The content of total sulfur impurities may be up to 5 ppm by weight, preferably up to 3 ppm by weight, in particular up to 2 ppm by weight, specifically up to 1 ppm by weight. Other impurities may be up to 2% by weight, preferably up to 0.5% by weight, in particular up to 0.1% by weight. Water may be present in the aromatic hydrocarbon or in the corresponding mixtures of aromatic hydrocarbons up to 0.1% by weight, preferably up to 0.07% by weight, in particular up to 0.05% by weight.

The desulfurization is carried out over a copper-zinc desulfurizing agent, if appropriate in the presence of hydrogen. This copper-zinc desulfurizing agent comprises at least copper and zinc, the copper:zinc atomic ratio being in the range from 1:0.3 to 1:10, preferably from 1:0.5 to 1:3 and in particular from 1:0.7 to 1:1.5. It is obtained by a coprecipitation process and can be used in oxidized or else in reduced form.

In a particular embodiment, the copper-zinc desulfurizing agent comprises at least copper, zinc and aluminum, the copper:zinc:aluminum atomic ratio being in the range from 1:0.3:0.05 to 1:10:2, preferably from 1:0.6:0.3 to 1:3:1 and in particular from 1:0.7:0.5 to 1:1.5:0.9.

The desulfurizing agent can be prepared by various processes. For example, an aqueous solution which comprises a copper compound, especially a water-soluble copper compound, for example copper nitrate or copper acetate, and a zinc compound, especially a water-soluble zinc compound, for example zinc nitrate or zinc acetate, together with an aqueous solution of an alkaline substance (for example sodium carbonate, potassium carbonate) can be mixed with one another to form a precipitate (coprecipitation process). The precipitate formed is filtered off, washed with water or first washed, then filtered and subsequently dried. Calcination is then effected at from about 270 to 400° C. Subsequently, the solid obtained is slurried in water, filtered off and dried. The copper-zinc desulfurizing agent thus obtained (“oxidized form”) can be used in the desulfurization in this form.

In a further embodiment, it is possible to subject the mixed oxide thus obtained to a hydrogen reduction. This is carried out at from about 150 to 350° C., preferably at from about 150 to 250° C., in the presence of hydrogen, the hydrogen being diluted by an inert gas, for example nitrogen, argon, methane, especially nitrogen, so that the hydrogen content is 10% by volume or less, preferably 6% by volume or less, in particular from 0.5 to 4% by volume. The copper-zinc desulfurizing agent thus obtained (“reduced form”) can be used in the desulfurization in this form.

In addition, the copper-zinc desulfurizing agent may also comprise metals which belong to group VIII of the Periodic Table (such as Fe, Co, Ni, Ru, Rh, Pd, Os, Ir, Pt), to group IB (such as Ag, Au) or to group VIB (such as Cr, Mo, W). These can be prepared by adding the appropriate metal salts to the abovementioned preparation processes.

It is also possible to shape or to extrude the solid obtained after the calcination or else that obtained after the hydrogen treatment to tablets or to other shapes, in which case it may be helpful to add additives, for example binders, for example graphite.

In a further embodiment, a solution which comprises a copper compound, especially a water-soluble copper compound, for example copper nitrate or copper acetate, a zinc compound, especially a water-soluble zinc compound, for example zinc nitrate or zinc acetate, and an aluminum compound, for example aluminum hydroxide, aluminum nitrate, sodium aluminate, together with an aqueous solution of an alkaline substance, for example sodium carbonate, potassium carbonate, can be mixed with one another to form a precipitate (coprecipitation process). The precipitate formed is filtered off, washed with water, or first washed, then filtered and dried. Calcination is then effected at from about 270 to 400° C. Subsequently, the solid obtained is slurried in water, filtered off and dried. The copper-zinc desulfurizing agent thus obtained (“oxidized form”) can be used in the desulfurization in this form.

In a further embodiment, it is possible to subject the mixed oxide thus obtained to a hydrogen reduction. This is carried out at from about 150 to 350° C., preferably at from about 150 to 250° C., in the presence of hydrogen, the hydrogen being diluted by an inert gas, for example nitrogen, argon, methane, in particular nitrogen, so that the hydrogen content is 10% by volume or less, preferably 6% by volume or less, in particular from 0.5 to 4% by volume. The copper-zinc desulfurizing agent thus obtained (“reduced form”) can be used in the desulfurization in this form.

In addition, the copper-zinc desulfurizing agent may also comprise metals which belong to group VIII of the Periodic Table (such as Fe, Co, Ni, Ru, Rh, Pd, Os, Ir, Pt), to group IB (such as Ag, Au) or to group VIB (such as Cr, Mo, W). These can be prepared by adding the appropriate metal salts to the abovementioned preparation processes.

It is also possible to shape or to extrude the solid obtained after the calcination or else that obtained after the hydrogen treatment to tablets or to other shapes, in which case it may be helpful to add additives, for example binders, for example graphite.

In a further embodiment, the coprecipitation can be carried out under pH control, for example, by adjusting the feed rate of the salt solutions such that a pH of from about 7 to 7.5 is maintained during the precipitation. It is also possible to subject the precipitate which is formed in the precipitation, after washing, to spray-drying.

In a further embodiment, the coprecipitation can be carried out in such a way that the copper oxide-zinc oxide components are precipitated from aqueous solutions of the corresponding salts (for example nitrates or acetates) with an alkaline substance (for example alkali metal carbonate, ammonium carbonate) in the presence of aluminum oxide, aluminum hydroxide in colloidal distribution (as a gel or sol).

The calcination, the hydrogen treatment which may be desired and the shaping can be effected as described above.

It is also possible to use commercially available catalysts, for example the catalyst R 3-12 from BASF or G-132A from Süd-Chemie.

In a preferred embodiment, the copper-zinc desulfurizing agent is used in reduced form. It may be advantageous to subject the mixed oxide which is obtained by the above-described processes to a hydrogen reduction which can be carried out as follows ([cat] hereinafter represents catalyst):
1. The mixed oxide is heated to from 100 to 140° C., in particular to 120±5° C., with a nitrogen stream of from 200 to 400 m³ (STP)/m³_{[CAT]}·h, in particular of 300±20 m³ (STP)/m³_{[CAT]}·h.
2. At the start of the reduction, 0.5±0.1% by volume of hydrogen is metered into the abovementioned nitrogen stream until a temperature increase of from 15 to 20° C. occurs and remains constant. Subsequently, the hydrogen stream is increased to 1.0±0.1% by volume of hydrogen until, overall, a temperature increase of max. 30±5° C. occurs and the temperature remains constant,
3. Subsequently, the hydrogen stream is increased to 2.0±0.2% by volume, but the temperature of the catalyst should not rise above 230° C., preferably 225° C.
4. The hydrogen stream is now increased to 4.0±0.4% by volume and the temperature of the nitrogen is simultaneously increased to 200±10°, but the temperature of the catalyst here too should not rise above 230° C., preferably 225° C.
5. The hydrogen stream is now increased to 6.0±0.6% by volume and the temperature of the catalyst is simultaneously kept at 220±10° C.
6. Subsequently, with a nitrogen stream of from 200 to 400 m³ (STP)/m³_{[CAT]}·h, in particular of 300±20 m³(STP)/m³_{[CAT]}·h, cooling is effected to below 50° C. at a cooling rate which should not exceed 50±5 K/h.

The copper-zinc desulfurizing agent thus obtained is then present in the reduced form and can be used thus. However, it can also be stored under inert gas until it is used. In addition, it is also possible to store the copper-zinc desulfurizing agent in an inert solvent. From case to case, it may be advantageous to store the copper-zinc desulfurizing agent in its oxidized form and to carry out the activation just in time. In this connection, it may also be advantageous to carry out a drying step before the activation. In this case, the calcined copper-zinc desulfurizing agent present in oxidic form is heated in a nitrogen stream of from 200 to 400 m³ (STP)/m³_{[CAT]}·h, in particular of 300±20 m³ (STP)/m³_{[CAT]}·h, to from 180 to 220° C., in particular to 200±10° C., at a heating rate which should not exceed 50 K/h. As soon as the water has been removed, cooling can be effected to from 100 to 140° C., in particular to 120±5° C., at a cooling rate which should not exceed 50 K/h, and the activation can be carried out as described above.

In an especially preferred embodiment, a copper-zinc desulfurizing agent is used which comprises from 35 to 45% by weight, preferably from 38 to 41% by weight, of copper oxide, from 35 to 45% by weight, preferably 38 to 41% by weight, of zinc oxide, and from 10 to 30% by weight, preferably from 18 to 24% by weight, of aluminum oxide, and if appropriate further metal oxides.

In an exceptionally preferred embodiment, a copper-zinc desulfurizing agent is used which comprises from 38 to 41% by weight of copper oxide, from 38 to 41% by weight of zinc oxide, and from 18 to 24% by weight of aluminum oxide.

These copper-zinc desulfurizing agents are obtainable from corresponding calcined mixed oxides by the abovementioned preparation processes.

In one embodiment, the desulfurization of the aromatic hydrocarbon or of the mixture of aromatic hydrocarbons, preferably of benzene, is carried out over the copper-zinc desulfurizing agent in oxidized form without addition of hydrogen.

In one further embodiment, the desulfurization of the aromatic hydrocarbon or of the mixture of aromatic hydrocarbons, preferably of benzene, is carried out over the copper-zinc desulfurizing agent in oxidized form in the presence of hydrogen.

In one further embodiment, the desulfurization of the aromatic hydrocarbon or of the mixture of aromatic hydrocarbons, preferably of benzene, is carried out over the copper-zinc desulfurizing agent in reduced form without addition of hydrogen.

In one further embodiment, the desulfurization of the aromatic hydrocarbon or of the mixture of aromatic hydrocarbons, preferably of benzene, is carried out over the copper-zinc desulfurizing agent in reduced form in the presence of hydrogen.

Typically, the desulfurization is carried out within a temperature range of from 40 to 200° C., particularly at from 50 to 180° C., in particular at from 60 to 160° C., preferably at from 70 to 120° C., at a pressure of from 1 to 40 bar, particularly at from 1 to 32 bar, preferably at from 1.5 to 5 bar, in particular at from 2.0 to 4.5 bar. The desulfurization may be carried out in the presence of inert gases, for example nitrogen, argon or methane. In general, however, the desulfurization is carried out without addition of inert gases.

Typically, if desired, hydrogen is used here which has a purity of ≧99.8% by volume, in particular of ≧99.9% by volume, preferably of ≧99.95% by volume. These purities apply analogously to the hydrogen which is used in the activations of the catalysts carried out if appropriate.

Typically the weight ratio of aromatic hydrocarbon or of the mixture of aromatic hydrocarbons to hydrogen is in the range from 40 000:1 to 1000:1, particularly in the range from 38 000:1 to 5000:1, especially in the range from 37 000:1 to 15 000:1, preferably in the range from 36 000:1 to 25 000:1, specifically in the range from 35 000:1 to 30 000:1.

In general, the LHSV (“Liquid Hourly Space Velocity”) is in the range from 0.5 to 10 kg of aromatic hydrocarbon per part by volume of catalyst and hour (kg/(m³_{[cat]}·h)), in particular in the range from 1 to 8 kg/(m³_{[cat]}·h), preferably in the range from 2 to 6 kg/(m³_{[cat]}·h).

The aromatic hydrocarbon or the mixture of aromatic hydrocarbons, preferably benzene, thus desulfurized now has a content of aromatic sulfur compounds of at most 70 ppb, preferably at most 50 ppb, and the total sulfur content is a total of 200 ppb, preferably ≦150 ppb, in particular ≦100 ppb.

The above-described desulfurizing agents also enable chlorine, arsenic and/or phosphorus or corresponding chlorine, arsenic and/or phosphorus compounds to be reduced or to be removed from the aromatic hydrocarbon or the mixture of aromatic hydrocarbons.

The aromatic hydrocarbon or the mixture of aromatic hydrocarbons can be desulfurized in one or more reactors connected in parallel or in series. These reactors are typically operated in liquid-phase mode, the gas and the liquid being conducted in cocurrent or in countercurrent, preferably in cocurrent. However, the possibility also exists of operating the reactors in trickle mode, the gas and the liquid being conducted in cocurrent or in countercurrent, preferably in countercurrent.

If necessary, the desulfurizing agent can also be removed again from the reactor. When the desulfurizing agent is present in reduced form, it may be advantageous to subject the desulfurizing agent to an oxidation before the deinstallation. The oxidizing agents used are oxygen or mixtures of oxygen with one or more inert gases, for example air. The oxidation is effected by customary processes known to those skilled in the art. For example, the oxidation can be carried out as follows:
1. The desulfurizing agent is first purged with a nitrogen stream of from 200 to 400 m³ (STP)/m³_{[CAT]}·h, in particular of 300±20 m³ (STP)/m³_{[CAT]}·h.
2. At the start of the oxidation, from 5 to 10 m³ (STP)/m³_{[CAT]}·h, in particular 7±1 m³ (STP)/m³_{[CAT]}·h, of air are metered into the abovementioned nitrogen stream, the temperature increasing to about 50° C. Subsequently, the air stream is increased to from 10 to 18 m³ (STP)/m³_{[CAT]}·h, in particular to 14±1 m³ (STP)/m³_{[CAT]}·h, within a period of from 0.5 h to 2 h, preferably 1±0.2 h, and maintained for from 6 to 10 h, preferably 8±0.5 h.
3. Subsequently, the air stream is increased to from 20 to 35 m³ (STP)/m³_{[CAT]}·h, in particular to 28±2 m³ (STP)/m³_{[CAT]}·h, over a period of from 0.5 h to 2.0 h, preferably 1±0.2 h, in the course of which the temperature of the desulfurizing agent should not rise above 230° C., preferably 225° C., and is maintained for from 3 to 5 h, preferably 4±0.5 h.
4. The air stream is then increased to from 120 to 180 m³ (STP)/m³_{[CAT]}·h, in particular to 150±10 m³ (STP)/m³_{[CAT]}·h, and the nitrogen stream is simultaneously lowered to likewise from 120 to 180 m³ (STP)/m³_{[CAT]}·h, in particular to 150±10 m³ (STP)/m³_{[CAT]}·h, in the course of which the temperature of the desulfurizing agent should not rise above 230° C., preferably 225° C. This method is continued until the temperature falls and the content of oxygen in the offgas corresponds to the starting content.
5. Subsequently, one nitrogen stream is reduced to zero and the air stream is increased to from 200 to 400 m³ (STP)/m³_{[CAT]}·h, in particular to 300±20 m³ (STP)/m³_{[CAT]}·h. This method is generally continued for about 1 hour until the oxidation is complete.
   The copper-zinc desulfurizing agent thus obtained can then be deinstalled.

In step b), the desulfurized aromatic hydrocarbon or the mixture of aromatic hydrocarbons is then hydrogenated in the presence of a supported ruthenium catalyst to the corresponding cycloaliphatics or the corresponding mixtures of cycloaliphatics, the catalyst having been applied to a support which has meso- and/or macropores.

The supports used may in principle be all supports which have macropores, i.e. supports which have exclusively macropores and also those which also comprise mesopores and/or micropores in addition to macropores. The terms “macropores”, “mesopores” and “micropores” are used in the context of the present invention as defined in Pure Appl. Chem. 46, 71 (1976), specifically as pores whose diameter is above 50 nm (macropores) or whose diameter is between 2 and 50 nm (mesopores) or whose diameter is <2 nm (micropores).

Especially suitable as supports are appropriate activated carbons, silicon carbides, aluminum oxides, silicon oxides, titanium dioxides, zirconium dioxides, or else mixtures thereof. Preference is given to using appropriate aluminum oxides, zirconium dioxides or silicon oxides, especially γ-aluminum oxide or silicon oxides.
In a particular embodiment, a γ-aluminum oxide-supported ruthenium catalyst is used.
   In general, the content of ruthenium is from 0.01 to 30% by weight, preferably from 0.01 to 5% by weight and in particular from 0.1 to 1.5% by weight, based in each case on the total weight of the catalyst.
   In a preferred embodiment, a supported ruthenium catalyst is used, the support having a mean pore diameter of at least 50 nm and a BET surface area of at most 30 m²/g and the amounts of ruthenium being from 0.01 to 30% by weight based on the total weight of the catalyst. Especially preferred are supported ruthenium catalysts, the support having a mean pore diameter of from 100 nm to 200 μm and a BET surface area of not more than 15 m²/g.
   In a further preferred embodiment, a supported ruthenium catalyst is used, the amounts of ruthenium being from 0.01 to 30% by weight based on the total weight of the catalyst, and from 10 to 50% of the pore volume of the support being formed by macropores having a pore diameter in the range from 50 nm to 10 000 nm and from 50 to 90% of the pore volume of the support being formed by mesopores having a pore diameter in the range from 2 to 50 nm and the sum of the fractions of the pore volumes adding up to 100%. (The mean pore diameter and the pore size distribution are determined by Hg porosimetry, in particular to DIN 66133.)
   The supported ruthenium catalysts are prepared by applying the ruthenium to the support. This can generally be done by impregnating the support with aqueous ruthenium salt solutions or by spraying the support with corresponding ruthenium salt solution. Suitable ruthenium salts are the nitrates, nitrosylnitrates, halides, carbonates, carboxylates, acetylacetonates, chlorine complexes, nitrito complexes or amine complexes, in particular the nitrate and the nitrosylnitrate.
   The supports coated or impregnated with the ruthenium salt solution are subsequently generally dried at temperatures of from 100 to 150° C. and optionally calcined at temperatures of from 200 to 600° C., preferably at from 350 to 450° C.
   The calcined, supported ruthenium catalyst thus obtained is then activated by treatment in a gas stream which comprises free hydrogen at temperatures of from 30 to 600° C., preferably at from 150 to 450° C. In general, the gas stream consists of from 50 to 100% by volume of hydrogen and up to 50% by volume of nitrogen.
   Typically, the ruthenium salt solution is applied to the support in such an amount that the content of ruthenium is from 0.01 to 30% by weight, preferably from 0.01 to 5% by weight, particularly from 0.01 to 1% by weight and especially from 0.05 to 1% by weight, based in each case on the total weight of the catalyst.
   In a particular embodiment, support materials are used which are macroporous and have a mean pore diameter of at least 50 nm, preferably of at least 100 nm, especially of at least 500 nm, and whose BET surface area is at most 30 m²/g, preferably at most 15 m²/g, particularly at most 5 m²/g and especially from 0.5 to 3 m²/g. The mean pore diameter of these supports is preferably from 100 nm to 200 μm, preferably from 500 nm to 50 μm (the surface area of the support is determined by the BET method by N₂ adsorption, in particular to DIN 66131).
   The metal surface area on the supported ruthenium catalyst thus obtained is from 0.01 to 10 m²/g, preferably from 0.05 to 5 m²/g and in particular from 0.05 to 3 m²/g. (The metal surface is determined by means of the chemisorption method described by J. Lemaire et al. in “Characterization of Heterogeneous Catalysts”, ed. Francis Delanney, Marcel Dekker, New York 1984, p. 310-324.)
   The ratio of the metal surface area to the catalyst surface area is at most 0.05, in particular at most 0.005.
   The pore distribution of the support may preferably be approximately bimodal. Such a bimodal pore diameter distribution preferably has maxima at about 600 nm and at about 20 μm.
   In a further preferred embodiment, support materials are used which have macropores and mesopores. In particular, they have such a pore distribution that from 5 to 50%, preferably from 10 to 45%, particularly from 10 to 30% and especially from 15 to 25% of the pore volume is formed by macropores having a pore diameter in the range from 50 nm to 10 000 nm, and from 50 to 95%, preferably from 55 to 90%, particularly from 70 to 90% and in particular from 75 to 85% of the pore volume is formed by mesopores having a pore diameter of from 2 to 50 nm. The sum of the fractions of the pore volumes adds up to 100%.
   The total pore volume of the supports used here is from 0.05 to 1.5 cm³/g, preferably from 0.1 to 1.2 cm³/g and especially from 0.3 to 1.0 cm³/g.
   The mean pore diameter of the supports used here is from 5 to 20 nm, preferably from 8 to 15 nm and especially from 9 to 12 nm. (The mean pore diameter is determined by Hg porosimetry, in particular to DIN 66133.)
   The surface area of the supports used here is in the range from 50 to 500 m²/g, preferably in the range from 200 to 350 m²/g and especially in the range from 200 to 300 m²/g. (The surface area of the support is determined by the BET method by N₂ adsorption, in particular to DIN 66131).
In a further embodiment, a coated catalyst comprising, as the active metal, ruthenium alone or together with at least one further metal of transition groups IB, VIIB or VIII of the Periodic Table of the Elements (CAS version), applied to a support comprising silicon dioxide as the support material may be used.
   In this coated catalyst, the amount of active metal is <1% by weight, preferably from 0.1 to 0.5% by weight, more preferably from 0.25 to 0.35% by weight, based on the total weight of the catalyst, and at least 60% by weight, more preferably 80% by weight of the active metal, based on the total amount of the active metal, is present in the coating of the catalyst up to a penetration depth of 200 μm. The aforementioned data are determined by means of SEM (scanning electron microscopy) EPMA (electron probe microanalysis)-EDXS (energy dispersive X-ray spectroscopy) and constitute average values. Further information regarding the aforementioned analysis methods and techniques are disclosed, for example, in “Spectroscopy in Catalysis” by J. W. Niemantsverdriet, VCH, 1995.
   In the coated catalyst, the predominant amount of the active metal is present in the coating up to a penetration depth of 200 μm, i.e. close to the surface of the coated catalyst. In contrast, only a very small amount of the active metal, if any, is present in the interior (core) of the catalyst.
   Preference is given to a coated catalyst in which no active metal can be detected in the interior of the catalyst, i.e. active metal is present only in the outermost coating, for example in a zone up to a penetration depth of 100-200 μm.
   In a further particularly preferred embodiment, a feature of the coated catalyst is that, in (FEG)-TEM (Field Emission Gun-Transmission Electron Microscopy) with EDXS, active metal particles can detect only in the outermost 200 μm, preferably 100 μm, most preferably 50 μm (penetration depth). Particles smaller than 1 nm cannot be detected.
   The active metal used may be ruthenium alone or together with at least one further metal of transition groups IB, VIIB or VIII of the Periodic Table of the Elements (CAS version). Suitable further active metals in addition to ruthenium are, for example, platinum, rhodium, palladium, iridium, cobalt or nickel or a mixture of two or more thereof. Among the metals of transition groups IB and/or VIIB of the Periodic Table of the Elements which can likewise be used, suitable metals are, for example, copper and/or rhenium. Preference is given to using ruthenium alone as the active metal or together with platinum or iridium in the coated catalyst; very particular preference is given to using ruthenium alone as the active metal.
   The coated catalyst exhibits the aforementioned very high activity at a low loading with active metal which is <1% by weight based on the total weight of the catalyst. The amount of the active metal in the coated catalyst is preferably from 0.1 to 0.5% by weight, more preferably from 0.25 to 0.35% by weight. It has been found that the penetration depth of the active metal into the support material is dependent upon the loading of the catalyst with active metal. Even in the case of loading of the catalyst with 1% by weight or more, for example in the case of loading with 1.5% by weight, a substantial amount of active metal is present in the interior of the catalyst, i.e. in a penetration depth of from 300 to 1000 μm, which impairs the activity of the hydrogenation catalyst, especially the activity over a long hydrogenation period, especially in the case of rapid reactions, where hydrogen deficiency can occur in the interior of the catalyst (core).
   In one embodiment of the coated catalyst, at least 60% by weight of the active metal, based on the total amount of the active metal, is present in the coating of the catalyst up to a penetration depth of 200 μm. In the coated catalyst, preferably at least 80% by weight of the active metal, based on the total amount of the active metal, is present in the coating of the catalyst up to a penetration depth of 200 μm. Very particular preference is given to a coated catalyst in which no active metal can be detected in the interior of the catalyst, i.e. active metal is present only in the outermost coating, for example in a zone up to a penetration depth of 100-200 μm. In a further preferred embodiment, 60% by weight, preferably 80% by weight, based on the total amount of the active metal, is present in the coating of the catalyst up to a penetration depth of 150 μm. The aforementioned data are determined by means of SEM (scanning electron microscopy) EPMA (electron probe microanalysis)-EDXS (energy dispersive X-ray spectroscopy) and constitute average values. To determine the penetration depth of the active metal particles, a plurality of catalyst particles (for example 3, 4 or 5) are abraded transverse to the extrudate axis (when the catalyst is present in the form of extrudates). By means of line scans, the profiles of the active metal/Si concentration ratios are then recorded. On each measurement line, a plurality of, for example 15-20, measurement points are measured at equal intervals; the measurement spot size is approx. 10 μm·10 μm. After integration of the amount of active metal over the depth, the frequency of the active metal in a zone can be determined.
   Most preferably, the amount of the active metal, based on the concentration ratio of active metal to Si, on the surface of the coated catalyst is from 2 to 25%, preferably from 4 to 10%, more preferably from 4 to 6%, determined by means of SEM EPMA-EDXS. The surface is analyzed by means of analyses of regions of 800 μm×2000 μm and with an information depth of approx. 2 μm. The elemental composition is determined in % by weight (normalized to 100%). The mean concentration ratio (active metal/Si) is averaged over 10 measurement regions.
   In the context of the present application, the surface of the coated catalyst is understood to mean the outer coating of the catalyst up to a penetration depth of approx. 2 μm. This penetration depth corresponds to the information depth in the aforementioned surface analysis.
   Very particular preference is given to a coated catalyst in which the amount of the active metal, based on the weight ratio of active metal to Si (wt./wt. in %), on the surface of the coated catalyst is from 4 to 6%, from 1.5 to 3% in a penetration depth of 50 μm and from 0.5 to 2% in the region of penetration depth from 50 to 150 μm, determined by means of SEM EPMA (EDXS). The values stated constitute averaged values.
   Moreover, the size of the active metal particles preferably decreases with increasing penetration depth, determined by means of (FEG)-TEM analysis.
   The active metal is present in the coated catalyst preferably partly or fully in crystalline form. In preferred cases, ultrafine crystalline active metal can be detected in the coating of the coated catalyst by means of SAD (Selected Area Diffraction) or XRD (X-Ray Diffraction).
   The coated catalyst may additionally comprise alkaline earth metal ions (M²⁺), i.e. M=Be, Mg, Ca, Sr and/or Ba, in particular Mg and/or Ca, most preferably Mg. The content of alkaline earth metal ion(s) (M²⁺) in the catalyst is preferably from 0.01 to 1% by weight, in particular from 0.05 to 0.5% by weight, very particularly from 0.1 to 0.25% by weight, based in each case on the weight of the silicon dioxide support material.
   An essential constituent of the catalysts is the support material based on silicon dioxide, generally amorphous silicon dioxide. In this context, the term “amorphous” is understood to mean that the fraction of crystalline silicon dioxide phases makes up less than 10% by weight of the support material. However, the support materials used to prepare the catalysts may have superstructures which are formed by regular arrangement of pores in the support material.
   Useful support materials are in principle amorphous silicon dioxide types which consist of silicon dioxide at least to an extent of 90% by weight, and the remaining 10% by weight, preferably not more than 5% by weight, of the support material may also be another oxidic material, for example MgO, CaO, TiO₂, ZrO₂, Fe₂O₃ and/or alkali metal oxide.
   In a preferred embodiment of the invention, the support material is halogen-free, especially chlorine-free, i.e. the content of halogen in the support material is less than 500 ppm by weight, for example in the range from 0 to 400 ppm by weight. Preference is thus given to a coated catalyst which comprises less than 0.05% by weight of halide (determined by ion chromatography) based on the total weight of the catalyst.
   Preference is given to support materials which have a specific surface area in the range from 30 to 700 m²/g, preferably from 30 to 450 m²/g (BET surface area to DIN 66131).
   Suitable amorphous support materials based on silicon dioxide are familiar to those skilled in the art and commercially available (see, for example, O. W. Flörke, “Silica” in Ullmann's Encyclopedia of Industrial Chemistry 6^{th} Edition on CD-ROM). They may be either of natural origin or have been synthetically produced. Examples of suitable amorphous support materials based on silicon dioxide are silica gels, kieselguhr, pyrogenic silicas and precipitated silicas. In a preferred embodiment of the invention, the catalysts have silica gels as support materials.
   Depending on the embodiment of the invention, the support material may have different shape. When the coated catalyst is used in fixed catalyst beds, use is typically made of moldings of the support material which are obtainable, for example, by extruding or tableting, and which may have, for example, the shape of spheres, tablets, cylinders, extrudates, rings or hollow cylinders, stars and the like. The dimensions of these moldings vary typically within the range from 0.5 mm to 25 mm. Frequently, catalyst extrudates with extrudate diameters of from 1.0 to 5 mm and extrudate lengths of from 2 to 25 mm are used. It is generally possible to achieve higher activities with smaller extrudates; however, these often do not have sufficient mechanical stability in the hydrogenation process. Very particular preference is therefore given to using extrudates with extrudate diameters in the range from 1.5 to 3 mm.
   The coated catalysts are prepared preferably by first impregnating the support material once or more than once with a solution of ruthenium(III) acetate alone or together with a solution of at least one further salt of metals of transition groups IB, VIIB or VIII of the Periodic Table of the Elements (CAS version), drying the resulting solid and subsequent reduction, the solution of the at least one further salt of metals of transition groups IB, VIIB or VIII of the Periodic Table of the Elements being applicable in one or more impregnation steps together with the solution of ruthenium(III) acetate or in one or more impregnation steps separately from the solution of ruthenium(III) acetate. The individual process steps are described in detail below.
   The preparation of the coated catalyst, comprising the steps of:
1) impregnating the support material comprising silicon dioxide once or more than once with a solution of ruthenium(III) acetate alone or together with a solution of at least one further salt of metals of transition groups IB, VIIB or VIII of the Periodic Table of the Elements (CAS version);
2) subsequent drying;
3) subsequent reduction;
the solution of the at least one further salt of metals of transition groups IB, VIIB or VIII of the Periodic Table of the Elements being applicable in one or more impregnation steps together with the solution of ruthenium(III) acetate or in one or more impregnation steps separately from the solution of ruthenium(III) acetate.
   In the abovementioned step 1), the support material comprising the silicon dioxide is impregnated once or more than once with a solution of ruthenium(III) acetate alone or together with at least one further dissolved salt of metals of transition groups IB, VIIB or VIII of the Periodic Table of the Elements (CAS version). Since the amount of active metal in the coated catalyst is very small, a simple impregnation is effected in a preferred embodiment. Ruthenium(III) acetate and the salts of metals of transition groups IB, VIIB or VIII of the Periodic Table of the Elements constitute active metal precursors. Especially in the case of use of ruthenium(III) acetate as a precursor, coated catalysts can be obtained which are notable, among other features, in that the significant portion of the active metal, preferably ruthenium alone, is present in the coated catalyst up to a penetration depth of 200 μm. The interior of the coated catalyst has only little active metal, if any.
   Suitable solvents for providing the solution of ruthenium(III) acetate or the solution of at least one further salt of metals of transition groups IB, VIIB or VIII of the Periodic Table of the Elements are water or else mixtures of water or solvents with up to 50% by volume of one or more water- or solvent-miscible organic solvents, for example mixtures with C₁-C₄-alkanols such as methanol, ethanol, n-propanol or isopropanol. Aqueous acetic acid or glacial acetic acid may likewise be used. All mixtures should be selected such that a solution or phase is present. Preferred solvents are acetic acid, water or mixtures thereof. Particular preference is given to using a mixture of water and acetic acid as a solvent, since ruthenium(III) acetate is typically present dissolved in acetic acid or glacial acetic acid. However, ruthenium(III) acetate may also be used as a solid after dissolution. The catalyst may also be prepared without use of water.
   The solution of the at least one further salt of metals of transition groups IB, VIIB or VIII of the Periodic Table of the Elements can be applied in one or more impregnation steps together with the solution of ruthenium(III) acetate or in one or more impregnation steps separately from the solution of ruthenium(III) acetate. This means that the impregnation can be effected with one solution which comprises ruthenium(III) acetate and also at least one further salt of metals of transition groups IB, VIIB or VIII of the Periodic Table of the Elements. The impregnation with this solution can be effected once or more than once. However, it is likewise possible that impregnation is effected first with a ruthenium(III) acetate solution and then, in a separate impregnation step, with a solution which comprises at least one further salt of metals of transition groups IB, VIIB or VIII of the Periodic Table of the Elements. The sequence of the impregnation steps may also be reversed. It is likewise possible that one of the two impregnation steps or both impregnation steps are repeated once or more than once in any sequence. Each impregnation step is typically followed by drying.
   Suitable salts of further metals of transition groups IB, VIIB or VIII of the Periodic Table of the Elements which can be used in the impregnation step are, for example, nitrates, acetonates and acetates, preference being given to acetates.
   Particular preference is given to effecting impregnation with a solution of ruthenium(III) acetate alone in one impregnation step.
   The impregnation of the support material can be effected in different ways and depends in a known manner upon the form of the support material. For example, the support material can be sprayed or flushed with the precursor solution or the support material can be suspended in the precursor solution. For example, the support material can be suspended in an aqueous solution of the active metal precursor and, after a certain time, filtered off from the aqueous supernatant. The amount of liquid absorbed and the active metal concentration of the solution can then be used to control the active metal content of the catalyst in a simple manner. The support material can also be impregnated by, for example, treating the support with a defined amount of the solution of the active metal precursor which corresponds to the maximum amount of liquid that the support material can absorb. For this purpose, the support material can, for example, be sprayed with the required amount of liquid. Suitable apparatus for this purpose is the apparatus used customarily for mixing liquids with solids (see Vauck/Müller, Grundoperationen chemischer Verfahrenstechnik [Basic operations in chemical process technology], 10th edition, Deutscher Verlag für Grundstoffundustrie, 1994, p. 405 ff.), for example tumble driers, impregnating drums, drum mixers, paddle mixers and the like. Monolithic supports are typically flushed with the aqueous solutions of the active metal precursor.
   The solutions used for impregnation are preferably low-halogen, especially low-chlorine, i.e. they comprise no or less than 500 ppm by weight, especially less than 100 ppm by weight of halogen, for example from 0 to <80 ppm by weight of halogen based on the total weight of the solution.
   The concentration of the active metal precursor in the solutions depends, by its nature, upon the amount of active metal precursor to be applied and the absorption capacity of the support material for the solution and is <20% by weight, preferably from 0.01 to 6% by weight, more preferably from 0.1 to 1.1% by weight, based on the total mass of the solution used.
   In step 2), drying is performed. This can be effected by customary processes for drying solids while maintaining the upper temperature limits specified below. The maintenance of the upper limit of the drying temperatures is important for the quality, i.e. the activity, of the catalyst. Exceedance of the drying temperatures specified below leads to a distinct loss of activity. Calcination of the support at higher temperatures, for example above 300° C. or even 400° C., as the prior art proposes, is not only superfluous but also has a disadvantageous effect on the activity of the catalyst. To achieve sufficient drying rates, the drying is effected preferably at elevated temperature, preferably at ≦180° C., particularly at ≦160° C., and at least 40° C., in particular at least 70° C., especially at least 100° C., very particularly in the range from 110° C. to 150° C.
   The solid impregnated with the active metal precursor is dried typically under standard pressure, and the drying can also be promoted by employing reduced pressure. Frequently, the drying will be promoted by passing a gas stream over or through the material to be dried, for example air or nitrogen.
   The drying time depends, by its nature, upon the desired degree of drying and the drying temperature and is preferably in the range from 1 h to 30 h, preferably in the range from 2 to 10 h.
   The drying of the treated support material is preferably carried out to such an extent that the content of water or of volatile solvent constituents before the subsequent reduction makes up less than 5% by weight, in particular not more than 2% by weight, based on the total weight of the solid. The weight fractions specified relate to the weight loss of the solid, determined at a temperature of 160° C., a pressure of 1 bar and a time of 10 min. In this way, the activity of the catalysts used can be enhanced further.
   In step 3), the solid obtained after the drying is converted to its catalytically active form by reducing the solid at temperatures in the range of generally from 150° C. to 450° C., preferably from 250° C. to 350° C., in a manner known per se.
   For this purpose, the solid obtained after the drying is contacted with hydrogen or a mixture of hydrogen and an inert gas at the above-specified temperatures. The absolute hydrogen pressure is of minor importance for the result of the reduction and can, for example, be varied within the range from 0.2 bar to 1.5 bar. Frequently, the catalyst material is hydrogenated at standard hydrogen pressure in a hydrogen stream. Preference is given to effecting the reduction with movement of the solid, for example by reducing the solid in a rotary tube oven or a rotary sphere oven. In this way, the activity of the catalysts can be enhanced further. The hydrogen used is preferably free of catalyst poisons such as compounds comprising CO and S, for example H₂S, COS and others.
   The reduction can also be effected by means of organic reducing reagents such as hydrazine, formaldehyde, formates or acetates.
   After the reduction, the catalyst can be passivated in a known manner to improve the handling, for example by treating the catalyst briefly with an oxygen-containing gas, for example air, but preferably with an inert gas mixture comprising from 1 to 10% by volume of oxygen. It is also possible here to use CO₂ or CO₂/O₂ mixtures.
   The active catalyst may also be stored under an inert organic solvent, for example ethylene glycol.
   To prepare the coated catalyst, in a further embodiment, the active metal catalyst precursor, for example prepared as above or prepared as described in WO-A2-02/100538 (BASF AG), can be impregnated with a solution of one or more alkaline earth metal(II) salts.
   Preferred alkaline earth metal(II) salts are corresponding nitrates, especially magnesium nitrate and calcium nitrate.
   The preferred solvent for the alkaline earth metal(II) salts in this impregnation step is water. The concentration of the alkaline earth metal(II) salt in the solvent is, for example, from 0.01 to 1 mol/liter.
   For example, the active metal/SiO₂ catalyst installed in a tube is contacted with a stream of an aqueous solution of the alkaline earth metal salt. The catalyst to be impregnated may also be treated with a supernatant solution of the alkaline earth metal salt.
   This preferably results in saturation of the active metal/SiO₂ catalyst, especially of its surface, with the alkaline earth metal ion(s) taking place.
   Excess alkaline earth metal salt and unimmobilized alkaline earth metal ions is/are flushed from the catalyst (H₂O rinsing, catalyst washing).
   For simplified handling, for example installation in a reactor tube, the catalyst can be dried after the impregnation. For this purpose, the drying can be carried out, for example, in an oven at <200° C., for example at from 50 to 190° C., more preferably at <140° C., for example at from 60 to 130° C.
   This impregnation process can be carried out ex situ or in situ: ex situ means before installation of the catalyst into the reactor; in situ means in the reactor (after the catalyst installation).
   In one process variant, the catalyst can also be impregnated in situ with alkaline earth metal ions by adding alkaline earth metal ions, for example in the form of dissolved alkaline earth metal salts, to the solution of the aromatic substrate (reactant) to be hydrogenated. To this end, for example, the appropriate amount of salt is first dissolved in water and then added to the substrate dissolved in an organic solvent.
   In one variant, the catalyst can be used in the hydrogenation process in combination with the substrate to be hydrogenated, which comprises a solution containing alkaline earth metal ions. The content of alkaline earth metal ions in the substrate to be hydrogenated is generally from 1 to 100 ppm by weight, in particular from 2 to 10 ppm by weight.
   As a result of the preparation, the active metal is present in the catalysts in the form of a metallic active metal.
   As a result of the use of halogen-free, especially chlorine-free, active metal precursors and solvents in the preparation of the coated catalyst, the halide content, especially chloride content, of the coated catalysts is additionally below 0.05% by weight (from 0 to <500 ppm by weight, for example in the range of 0-400 ppm by weight), based on the total weight of the catalyst.
The chloride content is determined by ion chromatography, for example with the method described below.
   In a selected variant, it is preferred that the percentage ratio of the Q₂ and Q₃ structures determined by means of ²⁹Si solid-state NMR, Q₂/Q₃, is less than 25, preferably less than 20, more preferably less than 15, for example in the range from 0 to 14 or from 0.1 to 13. This also means that the degree of condensation of the silica in the support used is particularly high.
   The Qₙ structures (n=2, 3, 4) are identified and the percentage ratio is determined by means of ²⁹Si solid-state NMR.

Qₙ═Si(OSi)ₙ(OH)₄₋ₙ where n=1, 2, 3 or 4.
When n=4, Qₙ is found at −110.8 ppm, when n=3 at −100.5 ppm and when n=2 at −90.7 ppm (standard: tetramethylsilane) (Q₀ and Q₁ were not identified).
   The analysis is carried out under the conditions of magic angle spinning at room temperature (20° C.) (MAS 5500 Hz) with cross-polarization (CP 5 ms) and using dipolar decoupling of ¹H. Owing to the partial overlapping of the signals, the intensities are evaluated by means of line shape analysis. The line shape analysis was carried out with a standard software package from Galactic Industries, by calculating a least squares fit iteratively.
   The support material preferably does not comprise more than 1% by weight and in particular not more than 0.5% by weight and in particular <500 ppm by weight of aluminum oxide, calculated as Al₂O₃.
   Since the condensation of silica can also be influenced by aluminum and iron, the total concentration of Al(III) and Fe(II and/or III) is preferably less than 300 ppm by weight, more preferably less than 200 ppm by weight, and is, for example, in the range from 0 to 180 ppm by weight.
   The fraction of alkali metal oxide results preferably from the preparation of the support material and can be up to 2% by weight. Frequently, it is less than 1% by weight. Also suitable are alkali metal oxide-free supports (0 to <0.1% by weight). The fraction of MgO, CaO, TiO₂ or of ZrO₂ may make up to 10% by weight of the support material and is preferably not more than 5% by weight. However, also suitable are support materials which do not comprise any detectable amounts of these metal oxides (from 0 to <0.1% by weight).
   Because Al(III) and Fe(II and/or III) can give rise to acidic sites incorporated into silica, it is preferred that charge compensation is present in the carrier, preferably with alkaline earth metal cations (M²⁺, M=Be, Mg, Ca, Sr, Ba). This means that the weight ratio of M(II) to (Al(III)+Fe(II and/or III)) is greater than 0.5, preferably >1, more preferably greater than 3. (The roman numerals in brackets after the element symbol mean the oxidation state of the element.)

The hydrogenation of the desulfurized aromatic hydrocarbon or of the mixture of desulfurized aromatic hydrocarbons, preferably benzene, over the above-described supported ruthenium catalysts to the cycloaliphatics or the corresponding mixture of cycloaliphatics, preferably cyclohexane, in the presence of hydrogen, can be carried out in the liquid phase or in the gas phase. The hydrogenation process is preferably carried out in the liquid phase—generally at a temperature of from 50 to 250° C., preferably at from 60 to 200° C., in particular at from 70 to 170° C. The pressures used are in the range from 1 to 200 bar, preferably from 10 to 50 bar, in particular from 19 to 40 bar and especially from 25 to 35 bar.

Typically hydrogen with a purity of ≧99.8% by volume, in particular of ≧99.9% by volume, preferably of 99.95% by volume, is used in the hydrogenation.

More preferably, the aromatic hydrocarbon or the mixture of aromatic hydrocarbons is hydrogenated fully, full hydrogenation being understood to mean a conversion of the compound to be hydrogenated of generally >98%, preferably >99%, more preferably >99.5%, even more preferably >99.9%, in particular >99.99% and especially >99.995%.

Typically, the weight ratio of aromatic hydrocarbon or of the mixture of aromatic hydrocarbons to hydrogen is in the range from 8:1 to 5:1, preferably from 7.7:1 to 5.5:1, in particular from 7.6:1 to 6:1 and especially from 7.5:1 to 6.5:1.

The hydrogenation of the desulfurized aromatic hydrocarbon or mixtures of desulfurized aromatic hydrocarbons can be carried out in one reactor or in a plurality of reactors connected in series or parallel, which are preferably operated in trickle mode. In this case, the gas and the liquid are conducted in cocurrent or in countercurrent, preferably in cocurrent. However, it is also possible to operate the reactors connected in series in liquid-phase mode.

In general, the LHSV (“Liquid Hourly Space Velocity”) is in the range from 0.1 to 10 kg of aromatic hydrocarbon per part by volume of catalyst and hour (kg/(m³_{[cat]}·h)), preferably in the range from 0.3 to 1.5 kg/(m³_{[cat]}·h). The trickle density is typically in the range from 20 to 100 m³ of aromatic hydrocarbon per unit of cross-sectional area of the catalyst bed available for flow and hour (m³/m²·h), preferably in the range from 60 to 80 m³/m²·h.

It may be advantageous, in a first reactor, to achieve a conversion of aromatic hydrocarbon of from 95 to 99.5% and, in a downstream reactor, a degree of conversion of >99.9%, in particular >99.99%, preferably >99.995%. In such a case, the ratio of the volumes of the catalyst beds of main reactor to downstream reactor is generally in the range from 20:1 to 3:1, in particular in the range from 15:1 to 5:1.

In a further embodiment, the main reactor can be operated in circulation mode. The circulation ratio (ratio of feed in kg/h to recycle stream in kg/h) is typically in the range from 1:5 to 1:100, preferably in the range from 1:10 to 1:50, preferentially in the range from 1:15 to 1:35. It is also possible in this case to remove the heat formed in the reaction partially or fully by passing the recycle stream through a heat exchanger.

In a further embodiment, the postreactor may also be integrated into the main reactor.

From case to case, it may also become necessary to regenerate the hydrogenation catalyst owing to declining activity. This is done by the methods which are customary for noble metal catalysts such as ruthenium catalysts and are known to those skilled in the art. These include, for example, the treatment of the catalyst with oxygen as described in BE 882 279, the treatment with diluted, halogen-free mineral acids as described in U.S. Pat. No. 4,072,628 or the treatment with hydrogen peroxide, for example in the form of aqueous solutions with a content of from 0.1 to 35% by weight, or the treatment with other oxidizing substances, preferably in the form of halogen-free solutions. Typically, the catalyst will be flushed with a solvent, for example water, after the reactivation and before the reuse.

The reaction product obtained in the process, i.e. the cycloaliphatic or the mixture of corresponding cycloaliphatics, can be purified further in a step c).

In the case that the reactant used is an aromatic hydrocarbon and the corresponding cycloaliphatic is obtained, the resulting reaction product can be subjected to a purifying distillation in order to remove any by-products formed, such as low boilers relative to the corresponding cycloaliphatic, for example n-hexane and n-pentane, or else high boilers. When, for example, benzene is used as the reactant, the cyclohexane obtained may comprise as impurities, for example, n-hexane and n-pentane, which can be removed as low boilers. Possible high boilers may include methylcyclohexane which can likewise be removed by distillation. In the purifying distillation, the pure cyclohexane can be obtained via a side draw in the column, while the low boiler components are drawn off at the top and high boiler components at the bottom. Alternatively, the purification of the product can also be effected in a column with a dividing wall, in which case the pure cyclohexane is drawn off at the level of the dividing wall.

When the reactant used is a mixture of aromatic hydrocarbons, the individual components of the cycloaliphatic mixture formed are separated by distillation and any further impurities are removed by distillation.

The heat of reaction arising in the course of the exothermic hydrogenation can, if appropriate, in the event of appropriate selection of the pressure level of the distillation, be utilized to operate the evaporator of the distillation column. To this end, the hot reaction effluent can be introduced directly into the column evaporator or, if appropriate, a secondary medium can be heated (for example generation of steam) and introduced into the column evaporator.

The partial steps of the process and also the overall process can be carried out continuously, semicontinuously or discontinuously.

With the aid of the process according to the invention, it is thus possible to obtain hydrogenated products which comprise very low residual contents, if any, of the starting materials to be hydrogenated.

The present invention further relates to a process for desulfurizing an aromatic hydrocarbon which comprises aromatic sulfur compounds, if appropriate in the presence of hydrogen, as described above in step a).

### Regeneration Step

In hydrogenation processes in which the catalysts described above are used, deactivation is observed after a period of operation of the catalyst. Such a deactivated ruthenium catalyst can be brought back to the state of the original activity by flushing. The activity can be restored to >90%, preferably >95%, more preferably >98%, in particular >99%, most preferably >99.5%, of the original value. The deactivation is attributed to traces or residues of water adsorbed on the catalyst. This can surprisingly be reversed by flushing with inert gas. The regeneration method of the invention can thus also be referred to as drying of the catalyst or removal of water from this.

“Flushing” means that the catalyst is brought into contact with inert gas. Normally, the inert gas is then passed over the catalyst by means of suitable constructional measures known to those skilled in the art.

The flushing with inert gas is carried out at a temperature of from about 10 to 350° C., preferably from about 50 to 250° C., particularly preferably from about 70 to 180° C., most preferably from about 80 to 130° C.

The pressures applied during flushing are from 0.5 to 5 bar, preferably from 0.8 to 2 bar, in particular from 0.9 to 1.5 bar.

According to the invention, the treatment of the catalyst is preferably carried out using an inert gas. Preferred inert gases comprise nitrogen, carbon dioxide, helium, argon, neon and mixtures thereof. Nitrogen is most preferred.

In a particular embodiment of the invention, the inventive method of regeneration is carried out without removal of the catalyst in the same reactor in which the hydrogenation has taken place. The flushing of the catalyst according to the present invention is particularly advantageously carried out at temperatures and pressures in the reactor which correspond to or are similar to those in the hydrogenation reaction, resulting in only a very brief interruption of the reaction process.

According to the present invention, the flushing with inert gas is carried out at a volume flow of from 20 to 200 standard l/h, preferably at a volume flow of from 50 to 200 standard l/h per liter of catalyst.

The flushing with inert gas is preferably carried out for a time of from 10 to 50 hours, particularly preferably from 10 to 20 hours. For example, the calculated drying time of the catalyst bed of an industrial cyclohexane production plant having an assumed moisture content of 2 or 5% by weight is approximately 18 or 30 hours, respectively. The flushing according to the method of the invention can be carried out either in a downward direction (downflow mode) or in an upward direction (upflow mode).

The present invention further provides an integrated process for the hydrogenation of an aromatic hydrocarbon in the presence of a ruthenium catalyst having a catalyst regeneration step. In step a) of this process, the aromatic hydrocarbon or the mixture of aromatic hydrocarbons, each of which comprises aromatic sulfur compounds as an impurity, is desulfurized and hydrogenated in step b). Thereinafter the hydrogenating catalyst is regenerated by flushing with inert gas, as laid out above, until the original activity or part of the original activity is attained.

According to an embodiment of the invention the aromatic hydrocarbon is benzene. In a further embodiment the aromatic hydrocarbon is a mixture of benzene and Toluene or mixtures which comprise benzene and xylene or a xylene isomer mixture, or mixtures which comprise benzene, toluene and xylene or a xylene isomer mixture.

The method of the invention is also suitable for drying catalysts which have absorbed water during various procedures such as maintenance or storage.

The method of the invention is also suitable for drying catalysts which have absorbed water during various procedures such as maintenance or storage.

The invention will be illustrated hereinafter with reference to the examples adduced:

### Examples of the Desulfurization of the Aromatic Hydrocarbon or of the Mixture of Aromatic Hydrocarbons (Stage a)

The experiments were performed in continuous tubular reactors with internal thermoelements (Ø 6 mm), trace heating (heating mats) and liquid metering.

The desulfurizing agent used was the catalyst R 3-12 from BASF Aktiengesellschaft in the form of 5×3 mm tablets—referred to hereinafter as catalyst A.

The desulfurizing agent was dried in accordance with the above description. To this end, the desulfurizing agent was heated to 200±10° C. in a nitrogen stream of 300±20 m³ (STP)/m³_{[CAT]}·h at a heating rate not exceeding 50 K/h. As soon as the water had been removed, the desulfurizing agent was cooled to 120±5° C. at a cooling rate not exceeding 50 K/h. The drying procedure was effected in trickle mode (flow direction from the top downward).

In some cases, the desulfurizing agent was used in its reduced form. In this case, the desulfurizing agent was converted from its oxidized form to its reduced form with hydrogen in accordance with the description. To this end, the dried desulfurizing agent (in its oxidized form) was heated to 120±5° C. with a nitrogen stream of 300±20 m³ (STP)/m³_{[CAT]}·h. 0.5±0.1% by volume of hydrogen was then metered to the abovementioned nitrogen stream until a temperature increase of from 15 to 20° C. occurred and remained constant. Subsequently, the hydrogen stream was increased to 1.0±0.1% by volume of hydrogen until a temperature increase of max. 30±5° C. occurred overall and the temperature again remained constant. The hydrogen stream was then increased to 2.0±0.2% by volume, in the course of which the temperature of the catalyst did not rise above 225° C. The hydrogen stream was then increased to 4.0±0.4% by volume and the temperature of the nitrogen was simultaneously increased to 200±10°, in the course of which the temperature of the catalyst did not rise above 225° C. A further increase in the hydrogen stream then to 6.0±0.6% by volume led to a rise in the temperature of the catalyst to 220±10° C., which was maintained. After one hour, the catalyst was then cooled to below 50° C. with a nitrogen stream of 300±2 m³ (STP)/m³_{[CAT]}·h at a cooling rate not exceeding 50±5 K/h. The reaction procedure was effected in trickle mode (flow direction from the top downward).

The feedstock used was benzene with a purity of >99.95%.

The benzene used and the reaction effluents were analyzed by gas chromatography with reporting of GC area percentages (instrument: HP 5890-2 with autosampler; range: 4; column: 30 m DB1; film thickness: 1 μm; internal column diameter: 0.25 mm; sample volume: 5 μl; carrier gas: helium; flow rate: 100 ml/min; injector temperature: 200° C.; detector: FID; detector temperature: 250° C.; temperature program: 6 min at 40° C., 10° C./min to 200° C. for 8 min, total running time 30 min).

The total sulfur content in the benzene used and the reaction effluents were analyzed by Wickbold combustion by means of ion chromatography. To this end, from 4 to 6 g of the sample are mixed with acetone (Merck Suprasolv, item No. 1.0012.1000) in a ratio of 1:1 and then combusted in a hydrogen-oxygen gas flame in a Wickbold combustion apparatus. The combustion condensate is collected in an alkaline receiver which comprises 40 mmol of KOH (Merck Suprapure, item No. 1.050.020.500) (aqueous solution). The sulfate formed from the sulfur and collected in the receiver is determined by ion chromatography. (Ion chromatography system: modular system, from Metrohm; precolumn: DIONEX AG 12, 4 mm; separating column: DIONEX AS 12, 4 mm; eluent: 2.7 mM Na₂CO₃ (Merck Suprapure, item No. 1.063.950.500) and 0.28 mM NaHCO₃ (Riedel de Haen, p.A., item No. 31437); flow rate: 1 ml/min; detection: conductivity after chemical suppression; suppressor: e.g. MSM, from Metrohm).

### Example a1

100 ml of catalyst A which had been dried by the drying procedure outlined above were charged in oxidic form into the above-described tubular reactor (Ø 25 mm×40 cm), the catalyst having been embedded into an inert bed of V4A rings above and below the actual catalyst bed. The height of the actual catalyst bed was approx. 22 cm. The experiment was carried out in liquid-phase mode at a pressure of 20 bar, 30 l (STP) of nitrogen per h having been metered into the liquid stream in cocurrent during the experiment.

TABLE 1

| | | Catalyst | Feed | | Effluent | |
|---|---|---|---|---|---|---|
| Run | Temper- | loading | | Total | Total | Effluent |
| time | ature | g/ | Benzene | sulfur | sulfur | Benzene |
| H | ° C. | (ml · h) | g/h | mg/kg | mg/kg | GC area % |
| 187 | 140 | 0.50 | 50 | 0.4 | <0.1 | 99.9723 |
| 211 | 140 | 0.50 | 50 | 0.4 | <0.1 | 99.9733 |
| 220 | 140 | 2.00 | 200 | 0.4 | <0.1 | 99.9758 |
| 235 | 140 | 2.00 | 200 | 0.4 | <0.1 | 99.9755 |
| 245 | 140 | 2.00 | 200 | 0.4 | <0.1 | 99.9712 |
| 259 | 140 | 2.00 | 200 | 0.4 | <0.1 | 99.9671 |
| 355 | 120 | 2.00 | 200 | 0.4 | <0.1 | 99.9635 |
| 379 | 120 | 2.00 | 200 | 0.4 | <0.1 | 99.9646 |
| 386 | 120 | 2.00 | 200 | 0.4 | <0.1 | 99.9718 |
| 403 | 120 | 2.00 | 200 | 0.4 | <0.1 | 99.9756 |
| 427 | 120 | 2.00 | 200 | 0.4 | <0.1 | 99.9772 |
| 499 | 120 | 2.00 | 200 | 0.4 | <0.1 | 99.9742 |
| 523 | 120 | 2.00 | 200 | 0.4 | <0.1 | 99.9771 |
| 547 | 100 | 2.00 | 200 | 0.4 | <0.1 | 99.9772 |

The data compiled in Table 1 show clearly that the desulfurization of the benzene used can be carried out with catalyst A in oxidic form.

### Example a2

100 ml of catalyst A which had been dried in accordance with the drying procedure outlined above and reduced in accordance with the activation procedure outlined above were charged in reduced form into the above-described tubular reactor (Ø 25 mm×80 cm), the catalyst having been embedded into an inert bed of V4A rings above and below the actual catalyst bed. The height of the actual catalyst bed was approx. 22 cm. The experiment was carried out in liquid-phase mode at a pressure of 20 bar, a mixture of nitrogen and hydrogen having been metered into the liquid stream in cocurrent during the experiment.

TABLE 2

| | | | | | Feed | | Effluent | |
|---|---|---|---|---|---|---|---|---|
| | | Feed | | Catalyst | | Total | Total | Effluent |
| Run time | Temperature | N₂ | H₂ | loading | Benzene | sulfur | sulfur | Benzene |
| h | ° C. | l (STP)/h | l (STP)/h | g/(ml · h) | g/h | mg/kg | mg/kg | GC area % |
| 187 | 80 | 30 | 2 | 2.04 | 204 | 0.4 | <0.1 | 99.9575 |
| 197 | 80 | 30 | 2 | 2.04 | 204 | 0.4 | <0.1 | 99.9564 |
| 211 | 80 | 30 | 2 | 2.04 | 204 | 0.4 | <0.1 | 99.9587 |
| 307 | 80 | 30 | 2 | 0.35 | 35 | 0.4 | <0.1 | 99.9502 |
| 331 | 80 | 30 | 2 | 2.04 | 204 | 0.4 | <0.1 | 99.9547 |
| 341 | 80 | 30 | 2 | 2.04 | 204 | 0.4 | <0.1 | 99.9572 |
| 379 | 40 | 30 | 2 | 2.04 | 204 | 0.4 | <0.1 | 99.9698 |
| 451 | 40 | 8 | 2 | 0.51 | 51 | 0.4 | <0.1 | 99.9657 |
| 475 | 40 | 8 | 2 | 2.04 | 204 | 0.4 | <0.1 | 99.9723 |
| 499 | 40 | 0 | 2 | 2.04 | 204 | 0.4 | <0.1 | 99.9730 |
| 548 | 40 | 0 | 2 | 0.30 | 30 | 0.4 | <0.1 | 99.9702 |
| 595 | 40 | 0 | 2 | 0.30 | 30 | 0.4 | <0.1 | 99.9663 |

The data compiled in Table 2 show clearly that the desulfurization of the benzene used can be carried out with catalyst A in reduced form.

### Example a3

100 ml of catalyst A which had been dried in accordance with the drying procedure outlined above and which had been reduced in accordance with the activation procedure outlined above were charged in reduced form into the above-described tubular reactor (Ø 25 mm×40 cm), the catalyst having been embedded into an inert bed of V4A rings above and below the actual catalyst bed. The height of the actual catalyst bed was approx. 22 cm. The experiment was carried out in liquid-phase mode at a pressure of 20 bar, 2 l (STP) of hydrogen per h having been metered into the liquid stream in cocurrent during the experiment.

TABLE 3

| | | | Feed | | Effluent | | Effluent |
|---|---|---|---|---|---|---|---|
| | | Catalyst | | Total | Total | Effluent | Cyclo- |
| Run time | Temperature | loading | Benzene | sulfur | sulfur | Benzene | hexane |
| h | ° C. | g/(ml_{cat} · h) | g/h | mg/kg | mg/kg | GC area % | GC area % |
| 188 | 60 | 1.00 | 100 | 0.16 | <0.1 | 99.9445 | 0.0360 |
| 196 | 80 | 1.00 | 100 | 0.16 | <<0.1 | 99.9372 | 0.0424 |
| 284 | 80 | 1.00 | 100 | 0.16 | <0.1 | 99.9369 | 0.0403 |
| 292 | 40 | 1.00 | 100 | 0.16 | <0.1 | 99.9372 | 0.0389 |
| 390 | 40 | 1.00 | 100 | 0.16 | <0.1 | 99.9641 | 0.0190 |
| 406 | 80 | 1.00 | 100 | 0.16 | <0.1 | 99.9528 | 0.0271 |
| 526 | 120 | 1.00 | 100 | 0.16 | <0.1 | 99.9421 | 0.0369 |
| 622 | 120 | 2.00 | 200 | 0.16 | <0.1 | 99.9429 | 0.0361 |
| 870 | 80 | 2.00 | 200 | 0.16 | <0.1 | 99.9677 | 0.0134 |
| 886 | 80 | 2.00 | 200 | 0.38 | <0.1 | 99.9686 | 0.0137 |
| 934 | 80 | 2.00 | 200 | 0.38 | <0.1 | 99.9682 | 0.0130 |
| 1222 | 80 | 2.00 | 200 | 0.45 | <0.1 | 99.9688 | 0.0129 |
| 1270 | 80 | 2.00 | 200 | 0.45 | <0.1 | 99.9700 | 0.0129 |
| 1294 | 80 | 2.00 | 200 | 0.45 | <0.1 | 99.9687 | 0.0147 |
| 1038 | 80 | 2.00 | 200 | 0.41 | <0.1 | 99.9678 | 0.0139 |
| 1126 | 80 | 2.00 | 200 | 0.41 | <0.1 | 99.9695 | 0.0122 |
| 1414 | 80 | 2.00 | 200 | 0.41 | 0.10 | 99.9666 | 0.0172 |
| 1462 | 80 | 2.00 | 200 | 0.56 | 0.12 | 99.9696 | 0.0139 |
| 1558 | 100 | 2.00 | 200 | 0.56 | <0.1 | 99.9686 | 0.014 |
| 1562 | 100 | 2.00 | 200 | 0.56 | <0.1 | 99.9682 | 0.0147 |

The data compiled in Table 3 show clearly that the desulfurization of the benzene used can be carried out with catalyst A in reduced form even in extended operation. Moreover, the data show clearly that only very small amounts of benzene are reduced to cyclohexane.

After this extended experiment had ended, the spent catalyst was deinstalled and analyzed. To this end, the catalyst was oxidized slowly with a nitrogen/air mixture or with pure air at a temperature of approx. 25-30° C. The oxidized catalyst was deinstalled in ten separate fractions with approximately equal volumes, a sample was removed in each case and these were analyzed by elemental analysis. The result of the analysis is listed in Table 4. The samples are numbered in accordance with the flow direction (liquid-phase mode, fraction 1 at the bottom, fraction 10 at the top).

TABLE 4

| | Sulfur |
|---|---|
| | [ppm] |
| Unused catalyst A | 6 |
| Fraction 10 | 250 |
| Fraction 9 | 310 |
| Fraction 8 | 300 |
| Fraction 7 | 430 |
| Fraction 6 | 440 |
| Fraction 5 | 500 |
| Fraction 4 | 870 |
| Fraction 3 | 1100 |
| Fraction 2 | 1400 |
| Fraction 1 | 2400 |

In accordance with expectation, the catalyst fraction at the reactor inlet (fraction 1) has the highest sulfur concentration, while the lowest content is present in the last fraction (fraction 10).

### Example a4

50 ml of catalyst A which had been dried in accordance with the drying procedure outlined above and which had been reduced in accordance with the activation procedure outlined above were charged in reduced form into the above-described tubular reactor (Ø 25 mm×40 cm), the catalyst having been embedded into an inert bed of V4A rings above and below the actual catalyst bed. The height of the actual catalyst bed was approx. 11 cm. The experiment was carried out in liquid-phase mode at a pressure of 3 bar, 2 l (STP) of hydrogen per h having been metered into the liquid stream in cocurrent during the experiment.

TABLE 5

| | | | Benzene feed | | Effluent | | |
|---|---|---|---|---|---|---|---|
| | | Catalyst | | Total | Total | Effluent | Effluent |
| Run time | Temperature | loading | | sulfur | sulfur | Benzene | Cyclohexane |
| h | ° C. | g/(ml · h) | g/h | mg/kg | mg/kg | GC area % | GC area % |
| 198 | 80 | 5.60 | 280 | 0.4 | <0.1 | 99.9768 | 0.0089 |
| 222 | 80 | 5.60 | 280 | 0.38 | <0.1 | 99.9792 | 0.0093 |
| 294 | 80 | 1.80 | 90 | 0.17 | <0.1 | 99.9783 | 0.0095 |
| 318 | 80 | 5.60 | 280 | 0.17 | <0.1 | 99.9779 | 0.0103 |
| 342 | 80 | 5.60 | 280 | 0.17 | <0.1 | 99.9803 | 0.0080 |
| 390 | 80 | 5.60 | 280 | 0.17 | <0.1 | 99.9776 | 0.0108 |
| 486 | 80 | 5.60 | 280 | 0.17 | <0.1 | 99.9789 | 0.009 |
| 510 | 80 | 5.60 | 280 | 0.19 | <0.1 | 99.9766 | 0.0108 |
| 654 | 80 | 1.10 | 55 | 0.19 | <0.1 | 99.9754 | 0.0117 |
| 678 | 80 | 5.60 | 280 | 0.19 | <0.1 | 99.9786 | 0.0093 |
| 702 | 80 | 5.60 | 280 | 0.18 | <0.1 | 99.9781 | 0.0099 |
| 726 | 80 | 5.60 | 280 | 0.18 | <0.1 | 99.9812 | 0.0086 |
| 798 | 80 | 1.80 | 90 | 0.18 | 0.1 | 99.9811 | 0.0084 |
| 846 | 80 | 5.60 | 280 | 0.18 | <0.1 | 99.9822 | 0.0075 |
| 870 | 80 | 5.60 | 280 | 0.18 | <0.1 | 99.9819 | 0.0075 |
| 894 | 80 | 5.60 | 280 | 0.14 | 0.1 | 99.9786 | 0.0109 |
| 966 | 80 | 1.80 | 90 | 0.1 | <0.1 | 99.9813 | 0.0073 |
| 990 | 80 | 5.60 | 280 | 0.1 | <0.1 | 99.982 | 0.0061 |
| 1014 | 80 | 5.60 | 280 | 0.1 | <0.1 | 99.9821 | 0.0061 |
| 1038 | 80 | 5.60 | 280 | 0.1 | <0.1 | 99.9803 | 0.0089 |
| 1062 | 80 | 5.60 | 280 | 0.1 | 0.1 | 99.9772 | 0.0117 |

The data compiled in Table 5 show clearly that a desulfurization can be carried out at 3 bar, 80° C. and catalyst loading of >5 kg_{benzene}/l_{catalyst}·h.

### Example a5

A continuous tubular reactor (Ø 46 mm×3500 mm) was charged with 3700 ml of catalyst A, the catalyst having been embedded into an inert bed above and below the actual catalyst bed (800 ml and 500 ml respectively). The installed catalyst A was then dried and reduced in trickle mode in accordance with the procedure outlined in Table 6.

TABLE 6

| | Temperature | | | | | | |
|---|---|---|---|---|---|---|---|
| | Bottom of | Middle of | Top of | | Feeds | | |
| Time | catalyst | catalyst | catalyst | Preheater | H₂ | N₂ | |
| h | bed ° C. | bed ° C. | bed ° C. | ° C. | l (STP)/h | l (STP)/h | |
| 0 | 152 | 153 | 153 | 156 | 0 | 1500 | Drying |
| 8 | 193 | 193 | 194 | 199 | 0 | 1500 | |
| 11 | 203 | 203 | 203 | 207 | 0 | 1500 | |
| 28 | 201 | 201 | 201 | 205 | 0 | 1500 | |
| 33 | 102 | 102 | 104 | 104 | 15 | 1500 | Activation |
| 36 | 139 | 138 | 140 | 148 | 15 | 1500 | (reduction) |
| 40 | 149 | 149 | 150 | 150 | 15 | 1500 | |
| 44 | 149 | 149 | 149 | 149 | 30 | 1500 | |
| 48 | 149 | 154 | 149 | 151 | 30 | 1500 | |
| 52 | 150 | 149 | 150 | 150 | 30 | 1500 | |
| 64 | 197 | 197 | 197 | 200 | 60 | 1500 | |
| 85 | 215 | 215 | 215 | 218 | 90 | 1500 | |
| 91 | 220 | 220 | 220 | 221 | 90 | 1500 | |
| 92 | 193 | 193 | 193 | 196 | 90 | 1500 | |
| 96 | 71 | 71 | 72 | 69 | 90 | 1500 | |
| 120 | 45 | 45 | 45 | 45 | 90 | 1500 | |

Subsequently, desulfurization was carried out at a pressure of from 3 to 32 bar in liquid-phase mode.

TABLE 7

| | | | | | | | | | Benzene feed analysis | | |
|---|---|---|---|---|---|---|---|---|---|---|---|
| | Temperature | | | | | Feeds | | | Sulfur GC | | |
| Run | | | | pre- | Pres- | Ben- | | Catalyst | | Thio- | Total |
| time | bottom | Middle | top | heater | sure | zene | H₂ | loading | COS | phene | sulfur |
| h | ° C. | ° C. | ° C. | ° C. | bar | kg/h | l/h | kg/(l*h) | ppb | ppb | mg/kg |
| 134 | 77 | 79 | 80 | 81 | 20 | 8.0 | 10 | 2.2 | | | 0.19 |
| 296 | 78 | 79 | 80 | 82 | 25 | 8.0 | 5 | 2.1 | | | 0.34 |
| 440 | 77 | 79 | 80 | 82 | 28 | 8.0 | 17 | 2.2 | | | 0.20 |
| 512 | 77 | 79 | 80 | 82 | 31 | 8.0 | 25 | 2.2 | | | 0.18 |
| 752 | 78 | 79 | 80 | 82 | 32 | 8.0 | 30 | 2.2 | | | 0.20 |
| 872 | 77 | 79 | 80 | 82 | 32 | 8.0 | 29 | 2.2 | | | 0.2 |
| 944 | 77 | 79 | 80 | 82 | 32 | 8.0 | 30 | 2.2 | | | 0.20 |
| 1016 | 77 | 79 | 80 | 82 | 32 | 8.0 | 30 | 2.2 | | | 0.27 |
| 1912 | 77 | 79 | 80 | 82 | 32 | 8.0 | 30 | 2.2 | | | 0.28 |
| 1936 | 77 | 79 | 80 | 82 | 32 | 8.0 | 30 | 2.2 | | | 0.28 |
| 1960 | 78 | 79 | 81 | 82 | 32 | 8.0 | 30 | 2.2 | | | 0.14 |
| 2104 | 78 | 79 | 81 | 82 | 32 | 8.0 | 30 | 2.2 | | | 0.19 |
| 2200 | 78 | 79 | 80 | 82 | 32 | 8.0 | 30 | 2.2 | 43 | 435 | 0.20 |
| 2296 | 77 | 79 | 80 | 83 | 32 | 8.0 | 30 | 2.2 | 35 | 227 | 0.47 |
| 2536 | 78 | 79 | 80 | 83 | 32 | 8.0 | 30 | 2.2 | 48 | 301 | 0.53 |
| 3232 | 77 | 79 | 80 | 82 | 32 | 8.0 | 30 | 2.2 | 20 | 295 | 0.28 |
| 3436 | 77 | 79 | 80 | 82 | 3 | 8.0 | 5 | 2.2 | 32 | 73 | 0.23 |
| 4036 | 77 | 79 | 80 | 82 | 3 | 8.0 | 5.5 | 2.2 | <50 | 75 | 0.24 |
| 4444 | 77 | 79 | 80 | 82 | 3 | 8.0 | 5.2 | 2.2 | <50 | 210 | 0.19 |
| 4940 | 76 | 78 | 79 | 81 | 3 | 8.0 | 6 | 2.2 | <50 | 440 | 0.15 |
| 5892 | 77 | 78 | 79 | 81 | 3 | 8.0 | 7 | 2.2 | <50 | 190 | <0.1 |
| 6780 | 76 | 78 | 79 | 81 | 3 | 8.0 | 4.4 | 2.2 | <50 | 390 | 0.24 |
| 7188 | 76 | 78 | 80 | 82 | 3 | 8.0 | 5.3 | 2.2 | | | 0.14 |

| | | | Benzene effluent analysis | | | | |
|---|---|---|---|---|---|---|---|
| | Benzene feed analysis | | Sulfur GC | | | | |
| Run | GC | GC | | Thio- | Total | GC | GC |
| time | benzene | cyclohexane | COS | phene | sulfur | benzene | cyclohexane |
| h | area % | area-ppm | ppb | ppb | mg/kg | area % | area-ppm |
| 134 | 99.9613 | 156 | | | <0.1 | 99.8970 | 170 |
| 296 | 99.974 | 103 | | | <0.1 | 99.9651 | 182 |
| 440 | 99.9658 | 192 | | | <0.1 | 99.9510 | 213 |
| 512 | | | | | <0.1 | 99.9545 | 252 |
| 752 | | | | | <0.1 | 99.9442 | 353 |
| 872 | 99.9522 | 273 | | | <0.1 | 99.952 | 273 |
| 944 | | | | | <0.1 | 99.9498 | 305 |
| 1016 | | | | | <0.1 | 99.9553 | 254 |
| 1912 | 99.9784 | 107 | <50 | <50 | <0.1 | 99.9385 | 489 |
| 1936 | | | <50 | <50 | 0.10 | 99.9598 | 276 |
| 1960 | 99.9772 | 123 | <50 | <50 | <0.1 | 99.9441 | 435 |
| 2104 | 99.9787 | 111 | <50 | <50 | <0.1 | 99.9434 | 467 |
| 2200 | 99.986 | 47 | <50 | <50 | 0.13 | 99.9767 | 122 |
| 2296 | 99.9802 | 92 | <50 | <50 | <0.1 | 99.9741 | 149 |
| 2536 | 99.9812 | 77 | <50 | <50 | <0.1 | 99.9809 | 63 |
| 3232 | 99.9839 | 76 | <50 | <50 | <0.1 | 99.9829 | 83 |
| 3436 | 99.9807 | 106 | <50 | <50 | <0.1 | 99.9828 | 85 |
| 4036 | 99.9862 | 61 | | | <0.1 | 99.9835 | 57 |
| 4444 | 99.9785 | 107 | <50 | <50 | <0.1 | 99.9786 | 92 |
| 4940 | 99.9744 | 103 | | | <0.1 | 99.9743 | 99 |
| 5892 | 99.976 | 48 | <50 | <50 | <0.1 | 99.9774 | 67 |
| 6780 | 99.9671 | | <50 | <50 | <0.1 | 99.9755 | 81 |
| 7188 | 99.9564 | 70 | | | <0.1 | 99.9605 | 88 |

The results of Table 7 show clearly that the content of aromatic sulfur compounds can be lowered below 70 ppb.

### Examples of the Hydrogenation of the Aromatic Hydrocarbon or of the Mixture of Aromatic Hydrocarbons (Stage b)

### General Process Description 1 (GPD 1)

The experiment was performed in a continuous jacketed reactor, (Ø 12 mm×1050 mm) with three oil heating circuits distributed uniformly over the reactor length. The reactor was operated in continuous trickle mode with controlled liquid circulation (HPLC pump). The experimental plant was also equipped with a separator for separating gas and liquid phase with level control, offgas regulator, external heat exchanger and sampler. The hydrogen was metered under pressure control (in bar); the hydrogen used in excess was measured under quantitative control (in l (STP)/h); the benzene feedstock was metered via an HPLC pump. The product was discharged under level control via a valve. The temperature was measured with a thermoelement at the start (inlet) and at the end (outlet) of the reactor or of the catalyst bed. The benzene used had a total sulfur content of <0.1 mg/kg (detection by ion chromatography). The catalyst used was a meso-/macroporous Ru/Al₂O₃ catalyst with 0.47% by weight of Ru (catalyst B) or a mesoporous Ru/SiO₂ catalyst with 0.32% by weight of Ru (catalyst C). These were prepared as detailed in the description. For example, catalyst C can be prepared as follows:

0 kg of the SiO₂ support (D11-10 (BASF); 3 mm extrudates (No. 04/19668), water uptake of 0.95 ml/g, BET 135 m²/g) are initially charged in an impregnating drum and impregnated at 96-98% by weight water uptake. The aqueous impregnating solution comprises 0.176 kg of Ru as ruthenium acetate from Umicore, 4.34% by weight of Ru, batch 0255). The impregnated catalyst is dried without motion at an oven temperature of 145° C. down to a residual moisture content of approximately 1%. The reduction is effected with motion in hydrogen (approximately 75% H₂ in N₂, N₂ being employed as the purge stream; 1.5 m³ (STP)/h of H₂-0.5 m³ (STP)/h of N₂) with a moving bed at 300° C. and a residence time of 90 minutes (1-2 h). The passivation is effected in dilute air (air in N₂). The addition of air is controlled such that the temperature of the catalyst remains below 30-35° C. The finished catalyst C comprises 0.31-0.32% by weight of Ru.

This catalyst is described in detail below:

Support: BASF D11-10 SiO₂ support (3 mm extrudate)

Porosity of the shaped body: 0.95 ml/g
(water uptake determination by saturating the support with water and then determining the supernatant solution and, after the water has dripped off, the amount of water taken up. 1 ml of water=1 g of water).

Tapped density of the shaped

body: 467 g/l (up to shaped body diameter of 6 mm).
Determination of the ruthenium content: from 0.03 to 0.05 gram of the sample is admixed with 5 g of sodium peroxide in an alsint crucible and heated slowly on a hotplate. Subsequently, the bulk flux mixture is first melted over an open flame and then heated over a blow-torch flame until it glows red. The fusion has ended as soon as a clear melt has been attained. The cooled melt cake is dissolved in 80 ml of water, and the solution is heated to boiling (destruction of H₂O₂) and then, after cooling, admixed with 50 ml of 21% by weight hydrochloric acid. Afterward, the solution is made up to a volume of 250 ml with water. This sample solution is analyzed by ICP-MS for isotope Ru 99.
Ru dispersity: 90-95% (by CO sorption, assumed stoichiometric factor: 1; sample preparation: reduction of the sample at 200° C. for 30 min with hydrogen and subsequently flushed with helium at 200° C. for 30 min—analysis of the metal surface with pulses of the gas to be adsorbed in an inert gas stream (CO) up to saturation of chemisorption at 35° C. Saturation has been attained when no further CO is adsorbed, i.e. the areas of 3 to 4 successive peaks (detector signal) are constant and similar to the peak of an unadsorbed pulse. Pulse volume is determined precisely to 1%; pressure and temperature of the gas must be checked). (Method: DIN 66136)

### Surface Analysis-Pore Distribution

N₂ sorption to DIN 66131/DIN 66134 or Hg porosimetry to DIN 66133
N₂ sorption: BET 130-131 m²/g (DIN 66131)
   Mean pore diameter 26-27 nm (DIN 66134)
   Pore volume: 0.84-0.89 ml/g
Hg porosimetry (DIN 66133)
   BET 119-122 m²/g
   Mean pore diameter (4V/A) 28-29 nm
   Pore volume: 0.86-0.87 ml/g

### TEM:

The reduced catalyst C comprises at least partly crystalline ruthenium in the outermost zone (extrudate surface). In the support, ruthenium occurs in individual particles 1-10 nm (in places >5 nm): usually 1-5 nm. The size of the particles decreases from the outside inward.

Ruthenium particles are seen up to a depth of 30-50 micrometers below the extrudate surface. In this coating, ruthenium is present at least partly in crystalline form (SAD: selected area diffraction). The main portion of the ruthenium is thus in this coating (>90% within the first 50 μm).

### General Experimental Description 2 (GED2)

A heatable 1.2 l pressure vessel (internal diameter 90 mm, vessel height: 200 mm, made of stainless steel) with 4-blade beam sparging stirrer, baffles and an internal riser for sampling or for charging and emptying the pressure vessel is charged with the particular amount (volume or mass) of the catalyst used in a “catalyst basket” (made of stainless steel).

The pressure vessel is sealed for pressure testing and charged with 50 bar of nitrogen. Afterward, the pressure vessel is decompressed, evacuated with a vacuum pump and isolated from the vacuum pump, and feedstock or the feedstock solution is sucked into the vessel via the riser.

To remove residual amounts of oxygen, the vessel is successively charged at room temperature twice with 10-15 bar each time of nitrogen and twice with 10-15 bar each time of hydrogen and decompressed.

The stirrer is switched on, a stirrer speed of 1000 rpm is established and the reaction solution is heated to reaction temperature. The target temperature is attained after 15 minutes at the latest. Hydrogen is injected up to the particular target pressure within 5 minutes. The hydrogen consumption is determined and the pressure is kept constant at the particular target pressure.

The riser is used at regular intervals to take preliminary samples (to flush the riser) and samples of the reaction mixture for monitoring the progress of the reaction.

After the appropriate reaction time, the heater is switched off, the pressure vessel is cooled to 25° C., the elevated pressure is released slowly and the reaction mixture is emptied via the riser with slightly elevated pressure. Afterward, the pressure vessel is evacuated with a vacuum pump and isolated from the vacuum pump, and new feedstock or the feedstock solution is sucked into the vessel via the riser.

This method enables the same catalyst to be used more than once. The hydrogen used had a purity of at least 99.9-99.99% by volume (based on dry gas). Secondary constituents are carbon monoxide (max. 10 ppm by volume), nitrogen (max. 100 ppm by volume), argon (max. 100 ppm by volume) and water (max. 400 ppm by volume).

The benzene used and the reaction effluents were analyzed by gas chromatography with reporting of GC area percentages (instrument: HP 5890-2 with autosampler; range: 4; column: 30 m DB1; film thickness: 1 μm; internal column diameter: 0.25 mm; sample volume: 5 μl; carrier gas: helium; flow rate: 100 ml/min; injector temperature: 200° C.; detector: FID; detector temperature: 250° C.; temperature program: 6 min at 40° C., 10° C./min to 200° C. for 8 min, total run time 30 min).

The total sulfur content in the benzene used and the reaction effluents were analyzed by Wickbold combustion by means of ion chromatography. To this end, from 4 to 6 g of the sample are mixed with acetone (Merck Suprasolv, item No. 1.0012.1000) in a ratio of 1:1 and then combusted in a hydrogen-oxygen gas flame in a Wickbold combustion apparatus. The combustion condensate is collected in an alkaline receiver which comprises 40 mmol of KOH (Merck Suprapure, item No. 1.050.020.500) (in water). The sulfate formed from the sulfur and collected in the receiver is determined by ion chromatography.

(Ion chromatography system: modular system, from Metrohm; precolumn: DIONEX AG 12, 4 mm; separating column: DIONEX AS 12, 4 mm; eluent: 2.7 mM Na₂CO₃ (Merck Suprapure, item No. 1.063.950.500) and 0.28 mM NaHCO₃ (Riedel de Haen, p.A., item No. 31437); flow rate: 1 ml/min; detection: conductivity after chemical suppression; suppressor: e.g. MSM, from Metrohm).

### Example b1

### According to GPD 1

104 ml (63.9 g) of catalyst B were used for continuous hydrogenation at a hydrogen pressure of 32 bar, at an offgas rate of 1-5 l (STP)/h, a reactor input temperature of 88-100° C. and a feed/circulation ratio of 1:30. The results are compiled in Table 8.

### Example b2

### According to GPD 1

104 ml (63.9 g) of catalyst B were used for continuous hydrogenation at a hydrogen pressure of 19 bar, at an offgas rate of 1-5 l (STP)/h, a reactor input temperature of 88-100° C. and a feed/circulation ratio of 1:30. The results are compiled in Table 9.

### Example b3

### According to GPD 1

104 ml (45.0 g) of catalyst C were used for continuous hydrogenation at a hydrogen pressure of 32 bar, at an offgas rate of 1-5 l (STP)/h, a reactor input temperature of 88-100° C. and a feed/circulation ratio of 1:30. The results are compiled in Table 10.

TABLE 8

| | | | | Reactor | Reactor | | | | | | | Ethyl- | | |
|---|---|---|---|---|---|---|---|---|---|---|---|---|---|---|
| | | Ben- | | inlet | outlet | C5- | | Methyl- | | Cyclo- | Methyl- | cyclo- | | |
| Pres- | Run | zene | Circu- | temper- | temper- | Al- | n-Hex- | cyclo- | | hexane | cyclo- | pen- | Tolu- | |
| sure | time | feed | lation | ature | ature | kanes | ane | pentane | Benzene | [GC | hexane | tane | ene | Others |
| [bar] | [h] | [g/h] | [g/h] | [° C.] | [° C.] | [GC area-ppm] | | | | area %] | [GC area-ppm] | | | |
| | Feedstock | | | | | 9 | 0 | 14 | 99.9763 ^{a)} | 65 ^{b)} | 44 | 25 | 37 | 43 |
| 32 | 22 | 62 | 1860 | 92 | 130 | 20 | 134 | 34 | 303 | 99.9340 | 77 | 25 | 0 | 67 |
| 32 | 49 | 62 | 1860 | 90 | 129 | 23 | 163 | 33 | 296 | 99.9311 | 77 | 26 | 0 | 71 |
| 32 | 94 | 62 | 1860 | 90 | 129 | 20 | 172 | 35 | 187 | 99.9430 | 75 | 25 | 0 | 56 |
| 32 | 142 | 62 | 1860 | 90 | 129 | 20 | 171 | 35 | 239 | 99.9380 | 75 | 25 | 0 | 55 |
| 32 | 239 | 62 | 1860 | 90 | 129 | 21 | 173 | 34 | 292 | 99.9325 | 74 | 24 | 0 | 57 |
| 32 | 286 | 62 | 1860 | 90 | 129 | 20 | 174 | 34 | 322 | 99.9291 | 77 | 25 | 0 | 57 |
| 32 | 404 | 62 | 1860 | 89 | 128 | 19 | 173 | 34 | 355 | 99.9259 | 78 | 25 | 0 | 57 |
| 32 | 468 | 62 | 1860 | 90 | 128 | 22 | 201 | 36 | 258 | 99.9326 | 77 | 25 | 0 | 55 |
| 20 | 540 | 62 | 1860 | 90 | 126 | 23 | 202 | 36 | 300 | 99.9282 | 76 | 25 | 0 | 56 |
| 20 | 588 | 62 | 1860 | 90 | 129 | 28 | 213 | 36 | 331 | 99.9235 | 77 | 25 | 0 | 55 |
| 20 | 698 | 62 | 1860 | 90 | 130 | 22 | 200 | 35 | 348 | 99.9217 | 78 | 24 | 0 | 76 |

(^{a)} [GC area %];
^{b)} [GC area-ppm])

TABLE 9

| | | | | Reactor | Reactor | | | | | | | Ethyl- | | |
|---|---|---|---|---|---|---|---|---|---|---|---|---|---|---|
| | | Ben- | | inlet | outlet | C5- | | Methyl- | | Cyclo- | Methyl- | cyclo- | | |
| Pres- | Run | zene | Circu- | temper- | temper- | Al- | n-Hex- | cyclo- | | hexane | cyclo- | pen- | Tolu- | |
| sure | time | feed | lation | ature | ature | kanes | ane | pentane | Benzene | [GC | hexane | tane | ene | Others |
| [bar] | [h] | [g/h] | [g/h] | [° C.] | [° C.] | [GC area-ppm] | | | | area %] | [GC area-ppm] | | | |
| Feedstock | | | | | | 9 | 0 | 10 | 99.9526 ^{a)} | 150 ^{b)} | 115 | 63 | 35 | 92 |
| 19 | 3 | 62.5 | 1860 | 89 | 130 | 34 | 148 | 17 | 445 | 99.9060 | 133 | 46 | 0 | 134 |
| 19 | 16 | 62.5 | 1860 | 89 | 129 | 34 | 292 | 17 | 301 | 99.8975 | 152 | 65 | 0 | 181 |
| 19 | 24 | 53.6 | 1830 | 89 | 129 | 33 | 290 | 17 | 122 | 99.9204 | 153 | 65 | 0 | 133 |
| 19 | 40 | 53.6 | 1830 | 89 | 129 | 32 | 297 | 17 | 151 | 99.9169 | 151 | 65 | 0 | 135 |
| 19 | 48 | 53.6 | 1830 | 89 | 129 | 32 | 302 | 17 | 173 | 99.9147 | 151 | 64 | 0 | 131 |
| 19 | 64 | 53.6 | 1830 | 89 | 129 | 32 | 311 | 18 | 191 | 99.9126 | 149 | 64 | 0 | 127 |
| 19 | 72 | 53.6 | 1830 | 89 | 129 | 31 | 315 | 18 | 199 | 99.9118 | 149 | 63 | 0 | 125 |
| 19 | 122 | 53.6 | 1830 | 89 | 129 | 32 | 335 | 19 | 303 | 99.8994 | 148 | 63 | 0 | 125 |
| 19 | 136 | 53.6 | 1830 | 89 | 129 | 33 | 335 | 19 | 316 | 99.8978 | 149 | 63 | 0 | 126 |
| 19 | 144 | 53.6 | 1830 | 89 | 129 | 33 | 339 | 19 | 337 | 99.8950 | 150 | 64 | 0 | 127 |
| 19 | 160 | 53.6 | 1830 | 89 | 129 | 33 | 342 | 20 | 360 | 99.8925 | 149 | 63 | 0 | 128 |
| 19 | 164 | 53.6 | 1830 | 89 | 129 | 33 | 343 | 19 | 376 | 99.8907 | 151 | 64 | 0 | 126 |
| 19 | 168 | 53.6 | 1830 | 89 | 129 | 32 | 345 | 20 | 397 | 99.8888 | 148 | 3 | 0 | 127 |

(^{a)} [GC area %];
^{b)} [GC area-ppm])

TABLE 10

| | | | | Reactor | Reactor | | | | | | Methyl- | Ethyl- | | |
|---|---|---|---|---|---|---|---|---|---|---|---|---|---|---|
| | | Ben- | | inlet | outlet | C5- | | Methyl- | | Cyclo- | cyclo- | cyclo- | | |
| Pres- | Run | zene | Circu- | temper- | temper- | Al- | n-Hex- | cyclo- | | hexane | hex- | pen- | Tolu- | |
| sure | time | feed | lation | ature | ature | kanes | ane | pentane | Benzene | [GC | ane | tane | ene | Others |
| [bar] | [h] | [g/h] | [g/h] | [° C.] | [° C.] | [GC area-ppm] | | | | area %] | [GC area-ppm] | | | |
| Feedstock | | | | | | 9 | 0 | 8 | 99.9728 ^{a)} | 0 ^{b)} | 55 | 29 | 135 | |
| 32 | 82 | 63 | 1860 | 90 | 128 | 35 | 213 | 19 | 0 | 99.9475 | 195 | 30 | 0 | 33 |
| 32 | 177 | 63 | 1860 | 90 | 128 | 30 | 196 | 17 | 0 | 99.9499 | 194 | 30 | 0 | 34 |
| 32 | 296 | 63 | 1860 | 89 | 128 | 29 | 185 | 18 | 0 | 99.9512 | 195 | 30 | 0 | 31 |
| 32 | 416 | 63 | 1860 | 90 | 128 | 24 | 169 | 17 | 0 | 99.9526 | 196 | 31 | 0 | 37 |
| 32 | 512 | 63 | 1860 | 100 | 139 | 45 | 370 | 23 | 0 | 99.9299 | 197 | 31 | 0 | 35 |
| 32 | 680 | 63 | 1860 | 100 | 139 | 42 | 331 | 22 | 0 | 99.9344 | 194 | 30 | 0 | 37 |
| 32 | 802 | 63 | 1860 | 100 | 139 | 37 | 308 | 21 | 0 | 99.9375 | 195 | 31 | 0 | 33 |
| 32 | 921 | 63 | 1860 | 100 | 139 | 37 | 304 | 23 | 0 | 99.9371 | 197 | 31 | 0 | 37 |
| 32 | 993 | 63 | 1860 | 100 | 139 | 37 | 305 | 22 | 0 | 99.9438 | 144 | 24 | 0 | 30 |

(^{a)} [GC area %];
^{b)} [GC area-ppm])

The data compiled in Tables 8 to 10 show clearly that cyclohexane can be obtained with an excellent selectivity.

### Example b4

The hydrogenation plant consists of a storage tank for the desulfurized benzene, a reservoir vessel, a metering pump for benzene, a main reactor (Ø 45×2000 mm) with separator for separating gas and liquid, and regulator for level control, liquid circulation with pump and a heat exchanger for removing the heat of reaction formed, a postreactor (Ø 22 mm×1500 mm) with a separator for separating gas and liquid, and regulator for level control, and also a storage tank for the hydrogenation effluent. The main reactor and postreactor were each equipped with an internal thermoelement (Ø 6 mm in the main reactor, Ø 3 mm in the postreactor). Both reactors were operated in trickle mode. Liquid and gas were metered in cocurrent.

The main reactor was charged with 2700 ml (1870 g), the postreactor with 340 ml (229 g), of catalyst B. For insulation and trace heating, the main reactor was equipped with electrical heating mats. The postreactor was manufactured for an adiabatic operating mode and was provided with appropriate insulation. Above and below the catalyst, an inert bed was introduced (wire mesh rings of stainless steel).

The feedstock used was desulfurized benzene which had been prepared analogously to Example a4 or a5, and had a total sulfur content of <0.1 mg/kg.

The benzene and the cyclohexane were analyzed by gas chromatography with reporting of GC area % or GC area-ppm; the analyses were carried out without internal standard (instrument: HP 5890-2 with autosampler; range: 4; column: 30 m DB1; film thickness: 1 μm; internal column diameter: 0.25 mm; sample volume: 5 μl; carrier gas: helium; flow rate: 100 ml/min; injector temperature: 200° C.; detector: FID; detector temperature: 250° C.; temperature program: 6 min at 40° C., 10° C./min to 200° C. for 8 min, total run time 30 min).

The total sulfur content in the benzene used and the reaction effluents were analyzed by Wickbold combustion by means of ion chromatography. To this end, from 4 to 6 g of the sample are mixed with acetone (Merck Suprasolv, item No. 1.0012.1000) in a ratio of 1:1 and then combusted in a hydrogen-oxygen gas flame in a Wickbold combustion apparatus. The combustion condensate is collected in an alkaline receiver which comprises 40 mmol of KOH (Merck Suprapure, item No. 1.050.020.500) (aqueous solution). The sulfate formed from the sulfur and collected in the receiver is determined by ion chromatography.

(Ion chromatography system: modular system, from Metrohm; precolumn: DIONEX AG 12, 4 mm; separating column: DIONEX AS 12, 4 mm; eluent: 2.7 mM Na₂CO₃ (Merck Suprapure, item No. 1.063.950.500) and 0.28 mM NaHCO₃ (Riedel de Haen, p.A., item No. 31437); flow rate: 1 ml/min; detection: conductivity after chemical suppression; suppressor: e.g. MSM, from Metrohm).

In some cases, the sulfur content was determined by gas chromatography (detection limits in each case 50 ppb for COS and thiophene) (separating column: CP SIL88 (100% cyanopropylpolysiloxane), length: 50 m; film thickness: 0.2 μm; internal diameter: 0.25 mm; carrier gas: helium; initial pressure: 1.5 bar; split: on column (ml/min); septum purge: 5 ml/min; oven temperature: 60° C.; preheating time: 10 min; rate 1: 5° C./min; oven temperature 1: 200° C.; continued heating time 1: 10 min; rate 2: -; oven temperature 2: -; continued heating time 2: -; injector temperature: on column (° C.); detector temperature: 220° C.; injector: HP autosampler; injection volume: 1.0 μl; detector type: PFPD (flame photometer); GC method: % by weight method with external calibration; special features: ON-column injection and special flame photometer detector).

At the start, the plant was operated at 20 bar; the plant pressure was increased to 32 bar after 860 operating hours. In the downstream reactor, hydrogenation was effected up to full conversion; in the reaction effluent, virtually no benzene was detectable any longer.

TABLE 11

| | | Main reactor | | | | | | | Postreactor | | | |
|---|---|---|---|---|---|---|---|---|---|---|---|---|
| | | Reactor | Reactor | | | | | | Reactor | Reactor | | |
| | | inlet | outlet | Ben- | | | Benzene | Cyclo- | inlet | outlet | Benzene | Cyclo- |
| Run | Pres- | temper- | temper- | zene | Circu- | Offgas | [GC | hexane | temper- | temper- | [GC | hexane |
| time | sure | ature | ature | feed | lation | [l (STP)/ | area- | [GC | ature | ature | area- | [GC |
| [h] | [bar] | [° C.] | [° C.] | [g/h] | [kg/h] | h] | ppm] | area %] | [° C.] | [° C.] | ppm] | area %] |
| 18 | 20 | 82 | 120 | 750 | 22.5 | 20 | 0 | 99.9613 | 85 | 82 | 7 | 99.9618 |
| 41 | 20 | 90 | 122 | 750 | 22.5 | 20 | 0 | 99.9546 | 84 | 81 | 0 | 99.9556 |
| 65 | 20 | 90 | 122 | 750 | 22.5 | 20 | 0 | 99.9524 | 84 | 85 | 0 | 99.9523 |
| 693 | 20 | 90 | 123 | 1220 | 36.6 | 40 | 17 | 99.9461 | 88 | 88 | | |
| 813 | 20 | 90 | 124 | 1300 | 39.0 | 40 | 42 | 99.9468 | 91 | 88 | 0 | 99.9519 |
| 865 | 32 | 90 | 123 | 1300 | 39.0 | 40 | 63 | 99.9449 | 90 | 89 | 0 | 99.9515 |
| 884 | 32 | 90 | 124 | 1400 | 42.0 | 40 | 4 | 99.9627 | 92 | 89 | 0 | 99.9619 |
| 892 | 32 | 90 | 123 | 1400 | 42.0 | 40 | 0 | 99.9640 | 94 | 90 | 0 | 99.9636 |
| 1003 | 32 | 90 | 124 | 1520 | 45.6 | 40 | 58 | 99.9557 | 90 | 89 | 0 | 99.962 |
| 1099 | 32 | 90 | 124 | 1520 | 45.6 | 40 | 315 | 99.9286 | 90 | 89 | 0 | 99.9631 |
| 1337 | 32 | 90 | 124 | 1520 | 45.6 | 45 | 472 | 99.8979 | 90 | 89 | 0 | 99.9684 |
| 1502 | 32 | 90 | 125 | 1520 | 45.6 | 45 | 1555 | 99.8043 | 92 | 92 | 0 | 99.9702 |
| 1770 | 32 | 90 | 125 | 1620 | 48.6 | 45 | 2091 | 99.7552 | 90 | 91 | 0 | 99.9668 |
| 1986 | 32 | 90 | 125 | 1620 | 48.6 | 45 | 2559 | 99.7083 | 90 | 92 | 0 | 99.9702 |

The present results show that the process according to the invention enables benzene to be converted fully and cyclohexane to be obtained in high purities.

### Example b5

The experiment was carried out under the same conditions and in the same plant as described in Example b4). However, the main reactor was charged with 2700 ml (1218 g) of catalyst C and the postreactor with 340 ml (153 g) of catalyst C. In addition, the plant was operated at 32 bar from the start onward.

The results obtained here too show that virtually no benzene is detectable any longer in the reaction effluent.

Additionally metered into the feed after a run time of 5347 h were 4.3% by weight of toluene. The corresponding amounts of methylcyclohexane were found in the reaction effluent but no toluene.

TABLE 12

| | Main reactor | | | | | | | Postreactor | | | | |
|---|---|---|---|---|---|---|---|---|---|---|---|---|
| | Reactor | Reactor | | | | | | Reactor | Reactor | | | |
| | inlet | outlet | | | | | Cyclo- | inlet | outlet | | Cyclo- | |
| Run | temper- | temper- | Benzene | Circu- | | Benzene | hexane | temper- | temper- | Benzene | hexane | |
| time | ature | ature | feed | lation | Loading | [GC area- | [GC | ature | ature | [GC area- | [GC | Offgas |
| [h] | [° C.] | [° C.] | g/h | kg/h | kg_{benzene}/(l*h) | ppm] | area %] | [° C.] | [° C.] | ppm] | area %] | [l (STP)/h] |
| 12 | 84.8 | 97.1 | 810 | 48.6 | 0.3 | 0 | 99.9688 | 79.3 | 84.5 | 0 | 99.9689 | 80 |
| 36 | 85.0 | 97.6 | 810 | 48.6 | 0.3 | 0 | 99.9685 | 78.1 | 84.3 | 0 | 99.8679 | 80 |
| 204 | 85.0 | 116.4 | 1620 | 48.6 | 0.6 | 0 | 99.9649 | 86.9 | 87.6 | 0 | 99.9646 | 40 |
| 514 | 84.8 | 117.5 | 1620 | 48.6 | 0.6 | 0 | 99.9720 | 86.0 | 87.1 | 0 | 99.9717 | 40 |
| 1018 | 85.0 | 117.5 | 1620 | 48.6 | 0.6 | 0 | 99.9761 | 85.1 | 85.4 | 0 | 99.9751 | 40 |
| 1042 | 84.8 | 117.5 | 1620 | 48.6 | 0.6 | 0 | 99.9764 | 84.0 | 85.4 | 0 | 99.9749 | 40 |
| 1066 | 85.3 | 117.2 | 1620 | 48.6 | 0.6 | 0 | 99.9739 | 85.0 | 85.3 | 0 | 99.9748 | 40 |
| 1090 | 85.1 | 117.5 | 1620 | 48.6 | 0.6 | 0 | 99.9751 | 86.5 | 86.0 | 0 | 99.9751 | 40 |
| 1498 | 85.0 | 117.0 | 1620 | 48.6 | 0.6 | 0 | 99.9769 | 87.3 | 85.9 | 0 | 99.9769 | 20 |
| 2002 | 84.8 | 116.4 | 1620 | 48.6 | 0.6 | 0 | 99.9769 | 85.2 | 85.6 | 0 | 99.9767 | 20 |
| 2508 | 84.8 | 116.3 | 1620 | 48.6 | 0.6 | 0 | 99.9790 | 85.6 | 85.9 | 0 | 99.9790 | 20 |
| 3012 | 85.1 | 117.5 | 1620 | 48.6 | 0.6 | 0 | 99.9792 | 84.9 | 85.9 | 0 | 99.9789 | 20 |
| 3276 | 84.8 | 118.5 | 1620 | 45.6 | 0.6 | 0 | 99.9790 | 85.0 | 85.7 | 0 | 99.9787 | 20 |
| 3324 | 84.8 | 124.1 | 1620 | 38.9 | 0.6 | 0 | 99.9767 | 85.1 | 85.5 | 0 | 99.9763 | 20 |
| 3516 | 85.0 | 124.9 | 1620 | 38.9 | 0.6 | 0 | 99.9768 | 83.3 | 85.6 | 0 | 99.9767 | 20 |
| 4020 | 84.8 | 124.7 | 1620 | 38.9 | 0.6 | 0 | 99.9729 | 85.0 | 85.7 | 0 | 99.9727 | 20 |
| 4524 | 85.0 | 125.4 | 1620 | 38.9 | 0.6 | 0 | 99.9696 | 83.4 | 85.4 | 0 | 99.9694 | 20 |
| 5012 | 84.8 | 123.4 | 1620 | 38.9 | 0.6 | 0 | 99.9764 | 84.7 | 85.6 | 0 | 99.9767 | 20 |
| 5299 | 84.8 | 124.1 | 1620 | 38.9 | 0.6 | 0 | 99.9731 | 85.1 | 85.8 | 0 | 99.9729 | 20 |

### Example b6

### According to GED 2

750 ml of a 5% solution of benzene in cyclohexane were hydrogenated with 9.0 g (approx. 22 ml) of catalyst C at a temperature of 100° C. and a pressure of 20 bar with hydrogen. The catalyst was used repeatedly in five successive experiments. Samples were taken after reaction times of 10, 20, 30, 40, 60, 90, 120 and 180 minutes.

Table 13 lists the decrease in the benzene content over time. The mean values of the results of the five experiments and the maximum positive and negative deviation from the mean for the particular samples are evaluated. The benzene content was determined by means of GC analysis in GC area %.

TABLE 13

| | Benzene content | | |
|---|---|---|---|
| Reaction | (mean of the 5 | Maximum negative | Maximum positive |
| time | experiments) | deviation from | deviation from |
| [min] | [GC area %] | the mean | the mean |
| 0 (starting | 5.394 | −0.015 | +0.005 |
| solution) | | | |
| 10 | 3.728 | −0.520 | +0.343 |
| 20 | 2.647 | −0.669 | +0.367 |
| 30 | 1.655 | −0.718 | +0.509 |
| 40 | 0.943 | −0.851 | +0.562 |
| 60 | 0.100 | −0.097 | +0.159 |
| 90 | 0.002 | −0.002 | +0.003 |
| 120 | 0 | 0 | 0 |
| 180 | 0 | 0 | 0 |

### Example b7

### According to GED 2

750 ml of a 5% solution of benzene in cyclohexane were hydrogenated with 9.0 g (approx. 22 ml) of catalyst C at a temperature of 100° C. and a pressure of 32 bar with hydrogen. The catalyst was used repeatedly in five successive experiments. Samples were taken after reaction times of 10, 20, 30, 40, 60, 90, 120 and 180 minutes.

Table 14 lists the decrease in the benzene content over time. The mean values of the results of the five experiments and the maximum positive and negative deviation from the mean for the particular samples are evaluated. The benzene content was determined by means of GC analysis in GC area %.

TABLE 14

| | Benzene content | | |
|---|---|---|---|
| Reaction | (mean of the 5 | Maximum negative | Maximum positive |
| time | experiments) | deviation from | deviation from |
| [min] | [GC area %] | the mean | the mean |
| 0 (starting | 5.394% | 0 | 0 |
| solution) | | | |
| 10 | 3.005% | −0.529 | +1.074 |
| 20 | 1.263% | −0.713 | +1.176 |
| 30 | 0.399% | −0.321 | +0.503 |
| 40 | 0.080% | −0.072 | +0.164 |
| 60 | 0.002% | −0.001 | +0.001 |
| 90 | 0.001% | −0.000 | +0.001 |
| 120 | 0.001% | −0.001 | +0.001 |
| 180 | 0% | 0 | 0 |

### Example c

### Regeneration of a Hydrogenation Catalyst

### Example of the Production of the Ruthenium Catalyst

A mesoporous/macroporous aluminum oxide support in the form of 3-5 mm sphere having a total volume of 0.44 cm³/g, with 0.09 cm³/g (20% of the total pore volume) being formed by pores having a diameter in the range from 50 nm to 10 000 nm and 0.35 cm³/g (80% of the total pore volume) being formed by pores having a diameter in the range from 2 nm to 50 nm, a mean pore diameter in the region of 11 nm and a surface area of 286 m²/g was impregnated with an aqueous ruthenium(III) nitrate solution. The volume of solution taken up during impregnation corresponded approximately to the pore volume of the support used. The support impregnated with the ruthenium(III) nitrate solution was subsequently dried at 120° C. and activated (reduced) in a stream of hydrogen at 200° C. The catalyst produced in this way comprised 0.5% by weight of ruthenium, based on the weight of the catalyst. The ruthenium surface area was 0.72 m²/g, and the ratio of ruthenium surface area to support surface area was 0.0027.

### Example 1

### Sorption Studies

The affinity of the catalyst for water was determined by means of measurements of the sorption of water vapor on the catalyst produced as described above (0.5% Ru/γ-Al₂O₃).

It was found that the catalyst sorbs an amount of water of 5% even at relatively low vapor pressures of 30%. If only traces of water are present in the reactor or in the starting materials, this water can be sorbed on the catalyst.

### Example 2

### Operating Life Experiment in the Hydrogenation of Benzene

In a plant for the preparation of cyclohexane using a ruthenium/aluminum oxide catalyst comprising 0.5% of Ru on a γ-Al₂O₃ support, a steady decrease in the catalyst activity and an increasing benzene content in the product stream are observed. Further monitoring of the reaction during a catalyst operating life test shows that the residual benzene content downstream of the main reactor in the hydrogenation of benzene increases from a few hundred ppm to some thousands of ppm over a period of operation of about 3400 hours. A calculation indicates that introduction of 16 620 kg/h of benzene having a water content of from 30 to 50 ppm introduces 0.8 kg of water per hour into the plant. In addition to this, there are a further 3.5 kg/h of water originating from the hydrogen gas.

When the plant was shut down after 3394 hours of operation, the plant ran with a residual benzene content of 0.2% at a WHSV of 0.6 g_{benzene}/ml_{cat}·h. During shutdown, the plant was flushed with pressurized nitrogen at a temperature of 70-100° C. and then depressurized. After start-up, the plant gave a residual benzene content of from 0.01% to 0.04% at a WHSV of 0.6 g_{benzene}/ml_{cat}·h.

This observed effect of drying of the catalyst was verified again after 7288 hours of operation. At a WHSV of 0.9 g_{benzene}/ml_{cat}·h, the residual benzene content at the end of the plant was 0.2% and even rose to 0.56%. After shutdown of the plant, the catalyst was dried by means of 100 standard l/h of nitrogen at 110° C. for a period of 34 hours. After start-up of the plant at a WHSV of 0.6 g_{benzene}/ml_{cat}·h, the residual benzene content was from 0.03% to 0.07%, which can be attributed to a significant increase in the catalyst activity as a result of drying.

In both cases, drying of the catalyst led to a significantly higher catalyst activity which is close to or equal to the original catalyst activity.

### Example 3

### Examination of the Influence of Water on the Hydrogenation of Benzene

To simulate the influence of water on the hydrogenation of benzene using a ruthenium catalyst, series of autoclave experiments before and after saturation of the catalyst with water and after drying of the catalyst were carried out. A 5% strength solution of benzene in cyclohexane together with the ruthenium catalyst was placed in the pressure vessel, the mixture was heated to the reaction temperature of 100° C. and the course of the reaction at a hydrogen pressure of 32 bar was followed by regular sampling. The samples were subsequently analyzed by gas chromatography.

23 hydrogenation experiments were carried out, and the catalyst was subsequently placed in water. 13 further hydrogenation experiments were then carried out. The catalyst displayed a significantly lower but virtually constant activity. After drying of the catalyst in a stream of nitrogen at 100° C. in a reaction tube, 5 further experiments were carried out; the catalyst displayed a hydrogenation activity similar to that before saturation with water.

The experiments demonstrate that the activity of the ruthenium/aluminum oxide catalyst used decreases significantly after contact with water, but the catalyst can be reactivated again by drying in a stream of nitrogen and the initial activity can be virtually fully restored.

What is claimed is:

1. A process comprising:
providing a starting material comprising one or more aromatic hydrocarbons, and having an aromatic sulfur compound content and a total sulfur content;
reducing the aromatic sulfur compound content and the total sulfur content in the starting material; and
hydrogenating the one or more aromatic hydrocarbons in the presence of a supported ruthenium catalyst and hydrogen.

2. The process according to claim 1, wherein the aromatic sulfur compound content is reduced to ≦70 ppb, and wherein the total sulfur content is reduced to ≦200 ppb.

3. The process according to claim 1, wherein reducing the aromatic sulfur compound content and the total sulfur content in the starting material is carried out in the presence of a desulfurizing agent comprising copper and zinc in an atomic ratio of 1:0.3 to 1:10.

4. The process according to claim 3, wherein the desulfurizing agent comprises 35 to 45% by weight of copper oxide, 35 to 45% by weight of zinc oxide, and 10 to 30% by weight of aluminum oxide.

5. The process according to claim 3, wherein the desulfurizing agent comprises an oxidized desulfurizing agent.

6. The process according to claim 3, wherein the desulfurizing agent comprises a reduced desulfurizing agent.

7. The process according to claim 1, wherein reducing the aromatic sulfur compound content and the total sulfur content in the starting material is carried out at a temperature of 40 to 200° C. and a pressure of 1 to 40 bar.

8. The process according to claim 1, wherein the supported ruthenium catalyst has a ruthenium content of 0.01 to 30% by weight, based on a total weight of the catalyst.

9. The process according to claim 1, wherein the supported ruthenium catalyst comprises a silicon oxide support material.

10. The process according to claim 1, wherein the supported ruthenium catalyst comprises an aluminum oxide support material.

11. The process according to claim 1, wherein the supported ruthenium catalyst comprises a coated catalyst having a coating wherein at least 60% by weight of the catalytically active ruthenium in the coating is present up to a penetration depth of 200 μm.

12. The process according to claim 1, wherein hydrogenating the one or more aromatic hydrocarbons is carried out at a temperature of 50 to 250° C. and at a pressure of 1 to 200 bar.

13. The process according to claim 1, wherein the one or more aromatic hydrocarbons comprises benzene.

14. The process according to claim 1, further comprising purifying the hydrogenated one or more aromatic hydrocarbons.

15. The process according to claim 14, wherein purifying the hydrogenated one or more aromatic hydrocarbons comprises distilling the hydrogenated one or more aromatic hydrocarbons.

16. The process according to claim 1, wherein reducing the aromatic sulfur compound content and the total sulfur content in the starting material is carried out in the presence of a reduced form desulfurizing agent, at a pressure of 2 to 4.5 bar and at a temperature of 50 to 180° C.; and wherein the desulfurizing agent comprises 35 to 45% by weight of copper oxide, 35 to 45% by weight of zinc oxide, and 10 to 30% by weight of aluminum oxide; and wherein hydrogenating the one or more aromatic hydrocarbons is carried out at a pressure of 19 to 40 bar and at a temperature of 70 to 170° C.; and wherein the supported ruthenium catalyst comprises an aluminum oxide support material and has a ruthenium content of 0.01 to 30% by weight, based on a total weight of the catalyst.

17. The process according to claim 1, wherein reducing the aromatic sulfur compound content and the total sulfur content in the starting material is carried out in the presence of a reduced form desulfurizing agent, at a pressure of 2 to 4.5 bar and at a temperature of 50 to 180° C.; and wherein the desulfurizing agent comprises 35 to 45% by weight of copper oxide, 35 to 45% by weight of zinc oxide, and 10 to 30% by weight of aluminum oxide; and wherein hydrogenating the one or more aromatic hydrocarbons is carried out at a pressure of 19 to 40 bar and at a temperature of 70 to 170° C.; and wherein the supported ruthenium catalyst comprises a silicon oxide support material and has a ruthenium content of 0.01 to 30% by weight, based on a total weight of the catalyst.

18. The process according to claim 1, wherein reducing the aromatic sulfur compound content and the total sulfur content in the starting material is carried out in the presence of hydrogen.

19. The process according to claim 1, further comprising a catalyst regeneration, the catalyst regeneration comprising flushing the supported ruthenium catalyst with an inert gas such that the supported ruthenium catalyst regains at least a portion of its catalytic activity.

20. A process comprising:
providing a starting material comprising one or more aromatic hydrocarbons, and having an aromatic sulfur compound content and a total sulfur content;
reducing the aromatic sulfur compound content and the total sulfur content in the starting material; wherein reducing the aromatic sulfur compound content and the total sulfur content in the starting material is carried out in the presence of a desulfurizing agent comprising 35 to 45% by weight of copper oxide, 35 to 45% by weight of zinc oxide, and 10 to 30% by weight of aluminum oxide; and wherein the aromatic sulfur compound content is reduced to ≦70 ppb, and wherein the total sulfur content is reduced to ≦200 ppb.
